(19) United States
(12) Patent Application Publication
(10) Pub. No.: US 2021/0017500 A1
(43) Pub. Date: Jan. 21, 2021

(54) ANIMAL CELL STRAIN AND METHOD FOR USE IN PRODUCING GLYCOPROTEIN, GLYCOPROTEIN AND USE THEREOF

(71) Applicant: JIANGNAN UNIVERSITY, Binhu Wuxi (CN)

(72) Inventors: Morihisa FUJITA, Binhu Wuxi (CN); Zecheng JIN, Binhu Wuxi (CN); Toshiniko KITAJIMA, Binhu Wuxi (CN); Huijie ZHANG, Binhu Wuxi (CN); Xiaodong GAO, Binhu Wuxi (CN)

(73) Assignee: JIANGNAN UNIVERSITY, Binhu Wuxi (CN)

(21) Appl. No.: 16/608,497

(22) PCT Filed: Apr. 26, 2018
(86) PCT No.: PCT/CN2018/084551

§ 371 Date: Oct. 25, 2019

(30) Foreign Application Priority Data
Apr. 28, 2017 (CN) 201710293579.9

(51) Int. Cl. C12N 5/10; C12N 9/40; C12N 9/24; A61K 38/47

(52) U.S. Cl. CPC C12N 5/10; C12Y 302/01022; C12Y 302/01024; C12N 2510/02; C12N 9/2465; C12N 9/2488; A61K 38/47; C12Y 302/01113

(57) ABSTRACT

Provided are an animal cell strain for use in producing a glycoprotein which uses a high-mannose sugar chain as a main N-glycan structure, a method for use in producing a glycoprotein by using the cell strain, a glycoprotein produced by using the method, and a use thereof. At least two genes from among a Golgi mannosidase and an endoplasmic reticulum mannosidase gene of the cell strain are damaged or knocked out.

### TECHNICAL FIELD

The present invention relates to an animal cell strain and method for producing a glycoprotein, a glycoprotein and use thereof, and more particularly to an animal cell strain for producing a glycoprotein having high-mannose type sugar chain as main N-linked sugar chain structure, a method for producing a glycoprotein using the animal cell strain, a glycoprotein produced by the animal cell strain, and use of the glycoprotein.

### BACKGROUND

Glycoprotein, which, as a kind of important functional proteins in organisms, is structurally a complex carbohydrate composed of a polypeptide chain to which branched oligosaccharide chain(s) are covalently linked. The oligosaccharide chains are linked to the polypeptide chains mainly in the forms of: Asn residue binding type (also known as N-glycosidic bond type), O-Ser/Thr type, GPI anchor type and proteoglycan type. The present invention mainly relates to the production of a glycoprotein having N-glycosidic bond type sugar chain (also known as N-linked sugar chain), commonly known as N-glycan. N-glycosidic bond type sugar chain has a pentasaccharide core, mainly including three types of oligosaccharide chains: \textcircled{1} high-mannose type, composed of GlcNAc and mannose; \textcircled{2} complex type: in addition to GlcNAc and mannose, further comprising fructose, galactose, sialic acid, etc.; \textcircled{3} hybrid type, containing characteristics of both \textcircled{1} and \textcircled{2}. Among them, a high-mannose type sugar chain is a marker of a glycoprotein transported into the lysosome of a mammalian cell such as a human cell, and it has been found that a glycoprotein can no longer exert its intrinsic activity after said sugar chain is removed.

A variety of hydrolases are contained in the lysosome, most of which are glycoproteins having sugar chain(s) and can degrade substances such as proteins, mucopolysaccharides, glycolipids into small molecules to be provided to cells for recycling. These hydrolases are synthesized in the endoplasmic reticulum, and their sugar chains are modified in the Golgi apparatus, prior to being transported to the lysosome by means of the recognition of specific M6PR receptors (see FIGS. 1(a) and (b)). The modification to sugar chain in the Golgi apparatus is often carried out by adding the moiety N-acetylglucosamine-1-phosphate (GlcNAc-1-P) of UDP-N-acetylglucosamine (UDP-GlcNAc) at position 6 of Man in the core glycan to produce Man-6-P-1-GlcNAc, and then removing the part GlcNAc to form a glycoprotein having an acidic sugar chain, which is then transported into the lysosome via recognition of a specific M6PR receptor.

When the produced hydrolase cannot be normally transported to the lysosome due to abnormal metabolic pathway(s) or a gene that controls the lysosomal enzyme is mutated, the intermediate product(s) in the reaction chain of the enzyme cannot be normally degraded but accumulated in the lysosome, which causes the dysfunction of the cells, tissues and/or organs, resulting in the occurrence of a lysosomal storage disease (see FIG. 1(c)). For example, in patients with Fabry's disease, glycolipids, especially the intermediate product, globortriaosylceramide (Gb3), cannot be decomposed but accumulated in the lysosome of cells due to lack of alpha-galactosidase, thus threatening the patients' lives. At present, the therapies for lysosomal storage diseases mainly include: enzyme replacement therapy, chemotherapy, gene modification therapy at gene level and the like, and the most classical method is the enzyme replacement therapy (see FIG. 1(d)). Due to the presence of M6PR on the surface of the cell membrane, M6PR can recognize the sugar chain structure on a drug protein and bring the protein to the lysosome, so that the lysosomal storage disease can be alleviated by replacing the damaged intrinsic hydrolase with the normal hydrolase by M6PR.

However, the enzyme replacement therapy is still very limited in use, since the effects of existing drugs for enzyme replacement therapy, such as the currently commercially available drugs for Fabry's disease, Fabrazyme from the Genzyme (β-galactosidase) and Replagal from the Shire HGT (alpha-galactosidase), are not satisfactory, and for most lysosomal storage diseases, there is no effective treatment.

Therefore, one of the urgent problems to be solved at present is to provide an effective treatment method for various lysosomal storage diseases.

In addition, glycoproteins used as drugs are often produced by animal cells using methods such as genetic recombination, which however, has many disadvantages such as high cost, low yield and heterogeneous sugar chains. The heterogeneity of the protein due to the heterogeneity of the sugar chains is one important issue that must be solved so as to maintain the stability and quality of the drug. For example, cytokines such as erythropoietin and granulocyte colony-stimulating factor can be active in vitro only when it has a sialic acid-containing complex type sugar chain (Delorme, E. et al., Biochemistry, 1992. 31(41): p 9871-6.; Haas, R. and S. Murea, Cytokines Mol Ther, 1995. 1(4): p 249-70). Therefore, to construct an animal cell strain capable of producing homogeneous glycoproteins is one of the urgent problems to be solved in the field of biopharmaceutical production.

The current methods for modifying sugar chains, especially high-mannose type sugar chains, are not satisfactory up to now. Among the methods for producing glycoproteins having high-mannose type N-linked sugar chains, a method using a cell strain disrupting or knocking out the gene MGAT1 encoding N-acetylglucosamine transferase I (GnT-I) is used for producing glycoproteins (Chen, W. and P. Stanley, Glycobiology, 2003. 13(1): p 43-50.; Reeves, P J et al., Proc Natl Acad Sci USA, 2002. 99(21): p 13419-24). Although such cell strain can produce glycoproteins having N-linked sugar chains with Man5-GlcNAc2 as main structure, it cannot produce glycoproteins having N-linked sugar chains with Man9-GlcNAc2 or Man8-GlcNAc2 structure, or glycoproteins having mannose-6-phosphate structure, while a glycoprotein containing five-mannose sugar chains cannot react with UDP-N-acetylglucosamine to form an acidic sugar chain, thereby reducing the efficiency of binding to the M6PR receptor.

Among other methods for producing glycoproteins having high-mannose type N-linked sugar chains, a method employing an alpha-1,2-mannosidase inhibitor such as kifunensine and deoxynojirimycin is used for producing glycoproteins (Elbein, A D et al., J Biol Chem, 1990. 265(26): p 15599-605). However, the use of mannosidase inhibitor results in sugar chains of M9 form, and if the inhibitor is continuously used to culture cells for long term, the content of complex type sugar chains in the obtained sugar chains is very high, leading to unideal stability and safety of the glycoproteins.

The heterogeneity of glycoproteins due to the heterogeneity of sugar chains can adversely affect the production and use of the glycoproteins. Since M6PR specifically recognizes glycoproteins, when partial sugar chain structures in the glycoproteins are not high-mannose type or no sugar chain phosphorated at position 6 is present, the uptaking efficiency of M6PR for drugs is reduced, and thus the treatment efficiency is not high. In addition, when sugar chains are not homogeneous, the structure of the sugar chains may also cause the glycoproteins to be recognized as foreign antigenic substances by the body, thereby causing an immune reaction. For the safety of drug molecules, it is necessary to ensure the homogeneity of sugar chains as much as possible.

### SUMMARY OF THE INVENTION

In order to solve the above problems, the inventors of the present invention conducted repeated intensive studies and found that, by destroying or knocking out at least two genes of the Golgi mannosidase and endoplasmic reticulum mannosidase genes, a glycoprotein with a greatly reduced content of complex type sugar chains, excellent stability and safety, and high-mannose type sugar chain as main N-linked sugar chain structure can be obtained (see FIG. 19).

Accordingly, it is an object of the present invention to provide an animal cell strain of producing a glycoprotein having high-mannose type sugar chain as main N-linked sugar chain structure, and a method of producing a glycoprotein having high-mannose type sugar chain as main N-linked sugar chain structure, a glycoprotein prepared by the method, and use of the glycoprotein.

In particular, the invention relates to the following technical solutions.

1. An animal cell strain of producing a glycoprotein having high-mannose type sugar chain as main N-linked sugar chain structure, characterized in that, at least two genes of the Golgi mannosidase and endoplasmic reticulum mannosidase genes in the cell strain are destroyed or knocked out.

2. The animal cell strain according to the above item 1, wherein the high-mannose type sugar chain is at least one selected from the group consisting of Glc1-Man9-GlcNAc2, Man9-GlcNAc2, Man8-GlcNAc2, Man7-GlcNAc2, Man6-GlcNAc2 and Man5-GlcNAc2.

3. The animal cell strain according to the above item 1, wherein the cell strain is derived from a mammalian cell selected from the group consisting of human embryonic kidney cells (HEK293), Chinese hamster ovary cells (CHO), COS, 3T3, myeloma, BHK, HeLa and Vero, or an amphibian cell selected from the group consisting of Xenopus egg cells or an insect cell Sf9, Sf21 or Tn5.

4. The animal cell strain according to the above item 3, wherein the cell strain is derived from human embryonic kidney cells (HEK293) or Chinese hamster ovary cells (CHO).

5. The animal cell strain according to the above item 1, wherein the destroying is achieved by a gene destroying method targeting a Golgi mannosidase gene and/or an endoplasmic reticulum mannosidase gene,

the knockout is achieved by a gene knockout method targeting a Golgi mannosidase gene and/or an endoplasmic reticulum mannosidase gene.

6. The animal cell strain according to the above item 5, wherein the endoplasmic reticulum mannosidase is:

(a) a protein encoded by the DNA sequence as set forth in SEQ ID NO: 43,

(b) a protein having more than 20% homology with the amino acid sequence of the protein encoded by the DNA sequence as set forth in SEQ ID NO: 43 and having the endoplasmic reticulum mannosidase activity.

7. The animal cell strain according to the above item 5, wherein the Golgi mannosidase I is:

(a) a protein encoded by the DNA sequence st forth in SEQ ID NO: 44,

(b) a protein having more than 20% homology with the amino acid sequence of the protein encoded by the DNA sequence as set forth in SEQ ID NO: 44 and having the Golgi mannosidase I activity,

(c) a protein encoded by the DNA sequence as set forth in SEQ ID NO: 45,

(d) a protein having more than 20% homology with the amino acid sequence of the protein encoded by the DNA sequence as set forth in SEQ ID NO: 45 and having the Golgi mannosidase I activity,

(e) a protein encoded by the DNA sequence as set forth in SEQ ID NO: 46,

(f) a protein having more than 20% homology with the amino acid sequence of the protein encoded by the DNA sequence as set forth in SEQ ID NO: 46 and having the Golgi mannosidase I activity.

8. The animal cell strain according to the above item 1, wherein the Golgi mannosidase gene is selected from the group consisting of the Golgi mannosidase I genes MAN1A1, MAN1A2 and MAN1C1, and the endoplasmic reticulum mannosidase gene is the endoplasmic reticulum mannosidase gene MAN1B1.

9. The animal cell strain according to the above item 1, wherein two genes of the Golgi mannosidase I genes MAN1A1, MAN1A2 and MAN1C1 in the cell strain are knocked out.

10. The animal cell strain according to the above item 9, wherein the cell strain is a MAN1A1/A2 gene double knocked-out cell strain A1/A2-double-KO (deposit No.: CTCCC No: C201767).

11. The animal cell strain according to the above item 1, wherein three genes selected from the group consisting of the Golgi mannosidase I genes MAN1A1, MAN1A2, MAN1C1 and the endoplasmic reticulum mannosidase gene MAN1B1 in the cell strain are knocked out.

12. The animal cell strain according to the above item 11, wherein the cell strain is a MAN1A1/A2/B1 gene triple knocked-out cell strain A1/A2/B1-triple-KO (deposite No.: CTCCC No: C2016193).

13. The animal cell strain according to the above item 1, wherein the glycoprotein is a lysosomal enzyme or an antibody.

14. The animal cell strain according to the above item 13, wherein the lysosomal enzyme is human alpha-galactosidase or human lysosomal lipase.

15. A method for producing a glycoprotein having high-mannose type sugar chain as main N-linked sugar chain structure, characterized in that comprising using the animal cell strain according to the above items 1 to 14.

16. A glycoprotein having high-mannose type sugar chain as main N-linked sugar chain structure prepared by the method of the above item 15.

17. The glycoprotein according to the above item 16, wherein the glycoprotein is human alpha-galactosidase or human lysosomal lipase.

18. The use of the glycoprotein of the above item 16 in the preparation of a medicament for treating a lysosomal storage disease.

19. The use according to the above item 18, wherein the lysosomal storage disease is Fabry's disease.

20. The use according to the above item 18, wherein the lysosomal storage disease is Wolman's disease or cholesterol ester storage disease.

According to the present invention, a glycoprotein with a greatly reduced content of complex type sugar chains, excellent stability and safety of glycoprotein, and high-mannose type sugar chain as main N-linked sugar chain structure can be obtained, and the sugar chains of the glycoprotein is highly homogeneous.

### BRIEF DESCRIPTION OF THE DRAWINGS

FIG. 1 is a schematic diagram showing recognition and transport of a lysosomal hydrolase by M6PR in the body. FIGS. 1a and 1b show normal recognition and transport of a lysosomal hydrolase, FIG. 1c shows abnormal recognition and transport of a lysosomal hydrolase, and FIG. 1d shows a treatment by supplementing a lysosomal enzyme that is deficient in a lysosomal storage disease in vitro.

FIG. 2 is an agarose gel electrophoresis photograph of the MAN1A1 knockout, the size of the wild type band is 431 bp before the knockout and is 358 bp after the knockout.

FIG. 3 is an agarose gel electrophoresis photograph of the MAN1A2 knockout, the size of the wild type band before the knockout is 247 bp, and is 215 bp after the knockout, and it can be seen that there are three different types of bands for the double knocked-out cells.

FIG. 4 shows the results of sequencing single knocked-out cell MAN1A1KO24. The sequence in the figure is the MAN1A1 gene near the guide RNA. Below the DNA sequence is the encoded amino acid sequence, the guide RNA of the target sequence is in gray bold, and the PAM sequence is underlined.

FIG. 5 shows the sequencing results of the single knocked-out cell MAN1A2KO37 and double knocked-out cell D-KO35. The sequence in the figure is the MAN1A2 gene near the guide RNA. Below the DNA sequence is the encoded amino acid sequence, the guide RNA of the target sequence is in gray bold, and the PAM sequence is underlined. There are three variants of the double knocked-out cell sequence, wherein one has a removal between the target sequences, one has an insertion mutation of a 75 bp fragment and one base “A”, and the last one has an insertion mutation of a 207 bp fragment and two bases “GA”.

FIG. 6 shows the results of flow cytometry analysis of the sugar chains on the surface of the single and double knocked-out cells using lectin ConA-FITC and PHA-L4-FITC.

FIG. 7 shows the results of flow cytometry analysis of staining of bulk cells with the MAN1C1 or MAN1B1 gene knocked out from DKO cells using lectin PHA-L4-FITC to determine changes of the sugar chains on the cell surface.

FIG. 8 is an agarose gel electrophoresis photograph of PCR amplification of the genome of bulk cells with the MAN1C1 or MAN1B1 gene knocked out from DKO cells to determine the gene knockout efficiency.

FIG. 9 is an agarose gel electrophoresis photograph for verifying the knockout result of MAN1B1, the size of the wild type band is 310 bp before the knockout, and the size of the T-KO band is 262 bp after the knockout.

FIG. 10 shows the results of sequencing of T-KO cells.

FIG. 11 shows the results of flow analysis of the sugar chain changes on the surface of WT, MAN1A1KO, MAN1A2KO, D-KO and T-KO cells.

FIG. 12 shows the relative fluorescence intensity by calculating the Mean value of the results of staining with ConA-FITC lectin, in which P-value is calculated, which shows the change in the relative fluorescence intensity.

FIG. 13 shows the relative fluorescence intensity by calculating the Mean value of the result of staining with PHA-L4-FITC lectin, in which P-value is calculated, which shows the change in the relative fluorescence intensity.

FIG. 14 shows the results of MALDI-TOF mass spectrometry analysis of whole cell sugar chains in the wild-type cells, double knocked-out cells D-KO, and triple knocked-out cells T-KO. The N-linked sugar chain with sialic acid is amidated during sample processing.

FIG. 15 shows the results of analysis of the sugar chain change of the recombinant protein sHF-GLA by western blot, wherein the secreted sHF-GLA was precipitated and enriched by anti-DDDDK beads and eluted through DDDDK peptides, and finally the protein prepared was processed by PNGaseF or Endo-H for three hours and then detected.

FIG. 16 shows the results of analysis of the sugar chain change of the recombinant protein sHF-LIPA by western blot, wherein the secreted sHF-LIPA was precipitated and enriched by anti-DDDDK beads and eluted through DDDDK peptides, and finally the protein prepared was processed by PNGaseF or Endo-H for three hours and then detected.

FIG. 17 shows the results of MALDI-TOF mass spectrometry analysis of the sugar chains of LIPA expressed by the wild type cell and triple knocked-out cell T-KO strain.

FIG. 18 shows the results of MALDI-TOF mass spectrometry analysis of the sugar chains of IgG expressed by the wild type cell and triple knocked-out cell T-KO strain.

FIG. 19 is a schematic illustration of the inventive concept of the present invention.

FIG. 20 is a schematic diagram showing the sugar chain structure of the high-mannose type sugar chains Glc1-Man9-GlcNAc2, Man9-GlcNAc2, Man8-GlcNAc2, Man7-GlcNAc2, Man6-GlcNAc2, and Man5-GlcNAc2 in the present invention.

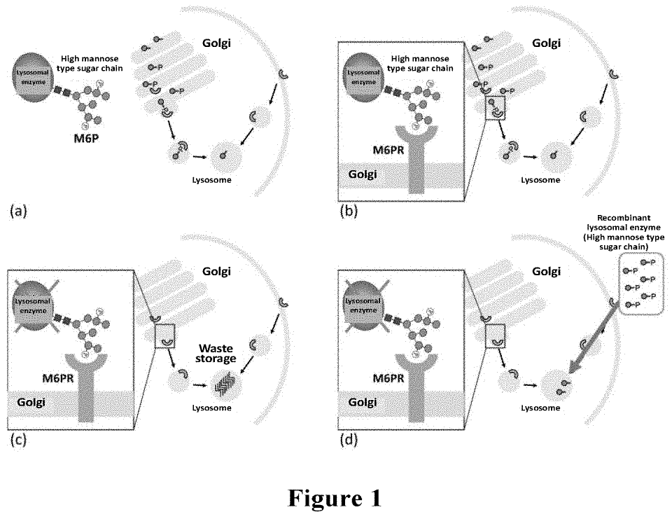

### DETAILED DESCRIPTION OF THE PREFERRED EMBODIMENTS

The embodiments of the present invention are described in detail below. It should be noted that these embodiments are merely exemplified for illustration purpose and are not intended to limit the scope of the present application.

One embodiment of the invention relates to an animal cell strain (hereinafter sometimes referred to as “cell strain of the invention”) of producing a glycoprotein (hereinafter sometimes referred to as “target protein”) having high-mannose type sugar chain as main N-linked sugar chain structure, wherein at least two genes of the Golgi mannosidase and endoplasmic reticulum mannosidase genes in the cell strain are destroyed or knocked out.

The inventors of the invention have conducted repeated studies on the synthesis of lysosomal hydrolases, sugar chain modification and the like, and found that by modifying at least two genes of the Golgi mannosidase I and endoplasmic reticulum mannosidase genes, a glycoprotein having high-mannose-type sugar chain, Man9-GlcNAc2 or Man9-GlcNAc2, as main N-linked sugar chain structure can be obtained. Thus, the present inventors have successfully constructed an animal cell strain of producing a glycoprotein having high-mannose type sugar chain as main N-linked sugar chain structure, wherein the animal cell strain is characterized in that at least two genes of the Golgi mannosidase I and endoplasmic reticulum mannosidase genes are destroyed or knocked out.

In the context of the invention, the wordings “Golgi mannosidase and/or endoplasmic reticulum mannosidase gene” may be simply referred to as “gene” or “target gene”, and they are used synonymously.

In the context of the invention, the wordings “glycoprotein having high-mannose type sugar chain as main N-linked sugar chain structure” means that the high-mannose type sugar chain accounts for more than 50%, preferably more than 60%, more than 70%, more preferably more than 80%, more than 90%, further preferably more than 95%, particularly preferably more than 98%, more than 99%, and most preferably 100% of the total sugar chains of the glycoprotein.

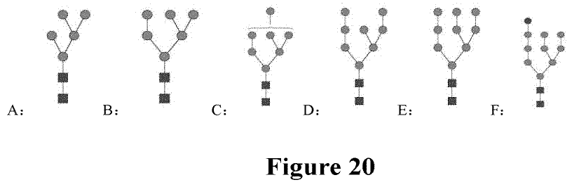

The high-mannose type sugar chain in the context of the invention refers to Glc1-Man9-GlcNAc2, Man9-GlcNAc2, Man8-GlcNAc2, Man7-GlcNAc2, Man6-GlcNAc2, and Man5-GlcNAc2, as well as the structures of these sugar chains containing a phosphorylation modification. Their sugar chain structures are shown in FIG. 20.

“Modification” in the present invention includes destroying and knocking out a gene.

In the context of the invention, “destroying a gene” means that the expression of a gene is inhibited by deletion, substitution, insertion and addition etc. to part of the base sequence of the gene (i.e., introduction of a mutation). Wherein, “inhibition of a gene expression” means that the amount of the expression of a potein normally encoded by a gene is reduced (i.e., the gene expression is partially inhibited), or a gene does not express a protein it normally encodes (i.e., the gene expression is completely inhibited), but “inhibition of a gene expression” is not limited to the case where the gene itself is not expressed, and may also include the case where a gene expresses itself but does not express a normal protein.

In the context of the invention, “knocking out a gene” is to delete a target gene in a chromosome. In the context of the invention, the wordings “knocking out a gene” and “inactivating a gene” are sometimes used synonymously. Among them, a cell in which a gene on a chromosome is destroyed by the CRISPR/Cas9 method or the like is considered as a gene knocked-out cell.

Generally, there are three Golgi mannosidase I genes (MAN1A1, MAN1A2 and MAN1C1) and one endoplasmic reticulum mannosidase gene (MAN1B1) on the chromosome of mammalian cells. MAN1A1 and MAN1A2 belong to the glycoside hydrolase family 47 (GH47) of the Carbohydrate-active enzyme database (CAZy). MAN1C1 and MAN1B1 are two other Golgi alpha-1,2-mannosidase and endoplasmic reticulum mannosidase gene belonging to the GH47 family.

In the cell strain of the present invention, at least two genes of the Golgi mannosidase I genes and the endoplasmic reticulum mannosidase gene on the chromosome are modified (destroyed or knocked out). By modification, the activity of the Golgi mannosidase and/or the endoplasmic reticulum mannosidase in the cell strain of the invention is reduced or eliminated.

The destroying is achieved by a gene destroying method targeting a Golgi mannosidase I gene and/or the endoplasmic reticulum mannosidase gene. Such a gene destroying method is, for example, a method of introducing a mutation into a Golgi mannosidase I gene and/or the endoplasmic reticulum mannosidase gene using a compound prone to generate a gene mutation such as ethyl methanesulfonate (EMS) and N-ethyl-N-nitrosourea (ENU).

The knockout is achieved by a gene knockout method targeting a Golgi mannosidase I gene and/or the endoplasmic reticulum mannosidase gene, and such a knockout method is, for example, a method of homologous interchange via gene manipulation or a method of editing the genome such as using CRISPR/Cas9.

In the cell strain of the present invention, preferably, at least two genes selected from the group consisting of the Golgi mannosidase I genes MAN1A1, MAN1A2, MAN1C1 and the endoplasmic reticulum mannosidase gene MAN1B1, more preferably at least two genes of the Golgi mannosidase I genes MAN1A1, MAN1A2 and MAN1C1 or at least one of the genes MAN1A1, MAN1A2 and MAN1C1 together with the endoplasmic reticulum mannosidase gene MAN1B1, are destroyed, and it is further preferred that the two genes MAN1A1 and MAN1A2, or the three genes MAN1A1, MAN1A2 and MAN1B1, are destroyed, and it is particularly preferred that the three genes MAN1A1, MAN1A2 and MAN1B1 are destroyed.

The MAN1A1/A2 gene-double-knocked-out cell strain A1/A2-double-KO (human embryonic kidney cell HEK293-MAN1A1&A2-DKO) obtained in the present invention was deposited in the China Center for Type Culture Collection (CCTCC) on Apr. 28, 2017. (Address: Wuhan University Depository Center, No. 299, Bayi Road, Wuchang District, Wuhan City, Hubei Province) with the deposit No.: CCTCC No: C201767.

The MAN1A1/A2/B1 gene-triple-knocked-out cell strain A1/A2/B1-triple-KO (human embryonic kidney cell HEK293-MAN1A1&A2&B1-TKO) obtained in the present invention was deposited in the China Center for Type Culture Collection (CCTCC) on Nov. 29, 2016 (Address: Wuhan University Depository Center, No. 299, Bayi Road, Wuchang District, Wuhan City, Hubei Province) with the deposit No.: CCTCC No: C2016193.

In the present invention, the endoplasmic reticulum mannosidase refers to:

(a) a protein encoded by the DNA sequence as set forth in SEQ ID NO: 43, or

(b) a protein having more than 20% homology with the amino acid sequence of the protein encoded by the DNA sequence as set forth in SEQ ID NO: 43 and having the endoplasmic reticulum mannosidase activity.

In the present invention, the Golgi mannosidase I refers to:

(a) a protein encoded by the DNA sequence as set forth in SEQ ID NO: 44,

(b) a protein having more than 20% homology with the amino acid sequence of the protein encoded by the DNA sequence as set forth in SEQ ID NO: 44 and having the Golgi mannosidase I activity,

(c) a protein encoded by the DNA sequence as set forth in SEQ ID NO: 45,

(d) a protein having more than 20% homology with the amino acid sequence of the protein encoded by the DNA sequence as set forth in SEQ ID NO: 45 and having the Golgi mannosidase I activity,

(e) a protein encoded by the DNA sequence as set forth in SEQ ID NO: 46, or

(f) a protein having more than 20% homology with the amino acid sequence of the protein encoded by the DNA sequence as set forth in SEQ ID NO: 46 and having the Golgi mannosidase I activity.

Human endoplasmic reticulum mannosidase is a protein encoded by the DNA sequence as set forth in SEQ ID NO: 43 (i.e., gene MAN1B1), and human Golgi mannosidase I is a protein endcoded by the DNA sequences of SEQ ID NOs: 44, 45 or 46 (i.e. the genes MAN1A1, MAN1A1 and MAN1C1, respectively).

Having more than 20% homology with the amino acid sequence of the protein encoded by the DNA sequence as set forth in SEQ ID NO: 43 means having more than 20% homology with the amino acid sequence of the human endoplasmic reticulum mannosidase encoded by the gene (MAN1B1), preferably having more than 30%, more than 40%, more than 50%, more than 60%, more than 70%, more than 80%, more than 90%, more than 95%, more than 98%, or more than 99% homology. Other expressions have similar meanings.

According to the present invention, a protein having high-mannose type sugar chain as main N-linked sugar chain structure can be obtained by expressing the protein using a cell strain in which a Golgi mannosidase I gene and the endoplasmic reticulum mannosidase gene on the chromosome are modified as a host cell.

Here, the host cell is not particularly limited, and various animal cells can be used, e.g. mammalian cells such as HEK293, CHO, COS, 3T3, myeloma, BHK, HeLa, and Vero; amphibian cells such as Xenopus egg cells or insect cells such as Sf9, Sf21 and Tn5, and the like. Among them, the Chinese hamster ovary cells (CHO) or human embryonic kidney cells (HEK293) are preferred, and the human embryonic kidney cells (HEK293) are particularly preferred.

The activity of the Golgi mannosidase and/or the endoplasmic reticulum mannosidase in these host cells is reduced or eliminated. A target protein having high-mannose type sugar chain as main N-linked sugar chain structure can be obtained by introducing an expression vector containing a gene encoding the target protein such as a lysosomal enzyme or an antibody to be produced into the host cells, or modifying the promoter of the gene on the chromosome. The expression vector of the encoding gene, for example, may be an expression vector such as pcDNA3, pEF, or pME originated from a mammal, from an animal virus, from a retrovirus, from an insect cell, from a plant, or the like. When the host cell is the HEK293 cell, it is preferred to use a mammalian-originated expression vector, an animal virus-originated expression vector, a retrovirus-originated expression vector, or a lentiviral-originated expression vector.

When a protein is expressed in animal cells such as HEK293, CHO, COS cells, etc., in order to express the protein in the cells, a necessary promoter is preferred, such as SV40 promoter, MMLV-LTR promoter, EF1alpha promoter, CMV promoter. Further, a drug resistance gene which can be used to screen according to the change in cell properties caused by a drug, such as neomycin, hygromycin, puromycin or blasticidin is preferred.

In addition, in order to stably express a gene in the cells, it is necessary to increase the copy number of the gene in the cells, for example, a corresponding vector having a DHFR gene (for example, pSV-dhfr) can be introduced into the CHO cells with the gene DHFR knocked-out, and the gene copy number is increased by using Methotrexate (MTX). In order to increase the gene copy number in the host cell strain, the expression vector, as a screening index, may also include a gene such as dihydrofolate reductase gene (dhfr), aminoglycoside transferase gene (APH), or thymidine kinase (TK) gene. Further, as for transient expression of a gene, a method empolying COS cells or HEK293 cells having a gene capable of expressing a SV40 T antigen on the chromosome and being transfected with a vector having a SV40 replication mechanism (such as pcDNA3) can be used. As the origin of replication, an origin originated from polyomavirus, adenovirus or Epstein-Barr virus and the like can be used.

The production method of a recombinant protein can be carried out by a method widely used in the art. In general, an appropriate expression vector having a protein-encoding gene is selected and introduced into a suitable host cell, and the transformant is recovered and cultured to obtain an extract or culture supernatant. The target protein can then be refined by separation through various chromatographic columns.

Another embodiment of the invention relates to a method of producing a glycoprotein having high-mannose type sugar chain as main N-linked sugar chain structure, characterized in that the above animal cell strain of the invention is used.

Another embodiment of the invention also relates to a glycoprotein having high-mannose type sugar chain as main N-linked sugar chain structure prepared by the method of the invention.

The glycoprotein of the invention is not particularly limited and may be various glycoproteins in the organisms, and preferred is a protein whose property such as protein activity, stability and intracellular uptake is altered with the sugar chain being converted to high-mannose-type N-linked sugar chain, e.g. a lysosomal enzyme or an antibody.

The lysosomal enzyme is not particularly limited, and may be various hydrolases in the lysosome, e.g. lipase or galactosidase.

Another embodiment of the invention also relates to the use of the glycoprotein of the invention in the manufacture of a medicament for the treatment of a lysosomal storage disease.

The lysosomal storage disease is not particularly limited, and examples thereof include mucopolysaccharidosis (a disease caused by an enzyme deficiency required for degradation of an acid mucopolysaccharide), Fabry's disease (also known as Sphingolipidosis, a pathology in central nervous system and other tissues resulting from storage of various sphingolipids such as cerebrosides, gangliosides or sphingomyelins in the lysosome due to deficiency in a lysosomal acid hydrolase, i.e. alpha-galactosidase, required for sphingolipid degradation, or lack of sphingolipid activator protein), Wolman's disease or cholesterol ester storage disease (storage of triglycerides and cholesterol esters in the lysosome, which is a disease caused by the lysosomal lipase deficiency), oligosaccharide storage disease (a disease caused by storage of various glycosides due to lack of a lysosomal acid hydrolase required for degradation of the carbohydrates in glycoproteins and glycolipids), and glycogen storage disease type II (caused by an acid alpha-glucosidase deficiency). Preferably, the lysosomal storage disease is Fabry's disease, Wolman's disease or cholesterol ester storage disease.

The glycoprotein of the invention can be directly administered to a patient in need thereof as a bioprotein drug, but it is usually preferred to administer a pharmaceutical composition containing one or more than two of these glycoproteins to a patient. As such a pharmaceutical composition, a formulation for oral administration such as tablet, capsule, granule, fine granule, powder, pill, troche, sublingual and liquid formulation, a formulation for parenteral administration such as injection, suppository, ointment, and patch.

The tablet or capsule for oral administration is usually provided in unit dosage, which can be manufactured by incorporating a commonly-used carrier for formulation such as a binder, a filler, a diluent, a compressor, a lubricant, a disintegrant, a colorant, a flavoring agent and a wetting agent. A tablet may be coated by a method known in the art, for example, using an enteric coating agent or the like, and may be produced using, for example, a filler, a disintegrator, a lubricant, a wetting agent, or the like.

The liquid formulation for oral administration can be provided in a form of dry preparation which can be reconstituted with water or a suitable medium before use, in addition to, for example, in a form of an aqueous or oily suspension, solution, emulsion, syrup or elixir. Such liquid formulation may be blended with a usual additive such as an anti-settling agent, an emulsifier, a preservative, and a usual flavoring or coloring agent as needed.

The formulation for oral administration can be produced by a method known in the art such as mixing, filling or tableting. Further, it is also possible to distribute the glycoprotein components to a formulation prepared by repeated formulating operations and using a large amount of a filler or the like.

The formulation for parenteral administration is usually provided in a dosage form of liquid carrier containing a glycoprotein as active ingredient and a sterile medium. The solvent for parenteral administration is usually produced by dissolving a substance used as active ingredient in a medium and sterilizing by filtering, followed by filling into a suitable vial or ampoule and sealing. To improve stability, the composition can be frozen, filled into vials, and the water is removed under vacuum. The parenteral suspension can be produced substantially in the same manner as the parenteral solution, but is preferably produced by suspending the active ingredient in a medium and sterilizing with ethylene oxide or the like. Further, in order to homogeneously distribute the active ingredient, a surfactant, a wetting agent, or the like may be added as needed. The invention is explained in more detail below by way of the examples, but these examples are not intended to limit the invention in any way.

### EXAMPLE 1

A double knocked-out cell strain of the Golgi a-mannosidase genes (mammalian cell strain: human embryonic kidney cell HEK293) was constructed using the CRISPR-Cas9 (Clustered Regularly Interspaced Short Palindromic Repeats) system.

1. Construction of the Plasmid for Knocking Out

Knocking out a gene using the CRISPR-Cas9 technology often requires designing a sequence fragment of 20 bp in length with a PAM site (NGG/NAG) behind the fragment. In this experiment, the gene sequences of two genes MAN1A1/MAN1A2 to be knocked out were obtained from NCBI (see SEQ ID NOs: 44 and 45, respectively). For the designing of guide-RNA, the DNA sequence of the guide-RNA required to knock out the gene was found on the Michael Boutros lab's Target Finder (http://www.e-crisp.org/E-CRISP/designcrispr.html).

The two target sequences of MAN1A1 and the primer sequences used were:

MAN1A1KO1:
(SEQ ID NO: 1)
AAAACCACGAGCGGGCTCTCAGG
Primer KO1F:
(SEQ ID NO: 2)
caccAAAACCACGAGCGGGCTCTC
Primer KO1R:
(SEQ ID NO: 3)
aaacGAGAGCCCGCTCGTGGTTTT
MAN1A1KO2:
(SEQ ID NO: 4)
CCACCTTCTTCTTCTCCAGTAGG
Primer KO2F:
(SEQ ID NO: 5)
caccCCACCTTCTTCTTCTCCAGT
Primer KO2R:
(SEQ ID NO: 6)
aaacACTGGAGAAGAAGAAGGTGG

The two target sequences of MAN1A2 and the primer sequences used were:

MAN1A2KO1:
(SEQ ID NO: 7)
CCTTTACCGGCATCTACATGTGG
Primer KO1F:
(SEQ ID NO: 8)
caccCCTTTACCGGCATCTACATG
Primer KO1R:
(SEQ ID NO: 9)
aaacCATGTAGATGCCGGTAAAGG
MAN1A2KO2:
(SEQ ID NO: 10)
CATGGATCAGGAAGACTCCGGGG
Primer KO2F:
(SEQ ID NO: 11)
caccCATGGATCAGGAAGACTCCG
Primer KO2R:
(SEQ ID NO: 12)
aaacCGGAGTCTTCCTGATCCATG

The plasmid pX330-EGFP containing the CRISPR-Cas9 system was cleaved with Bbs1 (NEB: R05395), and ligated with the DNA sequence of the designed guide-RNA using the Mighty Mix to construct the plasmid containing MAN1A1/MAN1A2 target sites and name them as follows:

pX330-EGFP-MAN1A1KO1/pX330-EGFP-MAN1A1KO2,
pX330-EGFP-MAN1A2KO1/pX330-EGFP-MAN1A2KO2.

2. Transfection

The wild type cells HEK293 were cultured overnight using 10% FCS medium and transfected when grown to approximately 90-95% confluent. The transfection reagent used was PEI-MAX (2 mg/ml pH 7.5), and the PEI-MAX was mixed with the OPTI (life technologies: 31985-070) evenly at a ratio of 1 μl PEI-MAX: 50 μl OPTI medium before transfection. The plasmid for knocking out and the plasmid pME-puro carrying the resistance gene were mixed homogeneously with the OPTI medium, and the ratio of the amount of the plasmid added was: 4 ug DNA: 5 μl PEI-MAX. The PEI-MAX solution and the plasmid-containing solution were mixed homogeneously and placed at room temperature for 25 minutes to allow the plasmid to bind to the PEI-MAX. The mixed solution was then added to the medium of the wild-type cell strain. The medium was replaced with fresh medium every 12 hours, and after the growth was resumed (about 24 hours), it was changed to a medium containing puromycin at a concentration of 1 μg/ml for screening.

3. Obtaining Single Colony and Verification

The selected cells contained the resistant plasmid and the plasmid for knocking out, and single cells were grown in a 96-well plate using limiting dilution to obtain single cell colonies. When the number of cells was increased, a single colony of cells was transferred to a 12-well plate culture. When it was grown to 100% confluency, the medium was removed, washed once with PBS, 100 μl of Tryp/EDTA was added to digest the cells, and 1 ml of the medium was added to harvest the cells. The obtained cell suspension was centrifuged at 3000 rpm for 2 min and rinsed again with 1 ml of PBS to obtain a pellet. 50 μl of 50 mM NaOH was added to the pellet and reacted at 95° C. for 20 min in a metal bath. After the reaction, 8.3 μl of 1M Tris (pH 7.5) was added and centrifuged at 15,000 rpm for 3 min, and the supernatant was taken for use.

The reaction system for the gene knockout results verification using KODFxNEO was as follows (10 μl):

The PCR reaction procedure is as follows:

| | |
|---|---|
| 5 μl | KOD buffer |
| 0.2 μl | KODF × NEO |
| 0.4 μl | primer F |
| 0.4 μl | primer R |
| 2 μl | dNTP |
| 1 μl | ddwater |
| 0.5 μl | DMSO |
| 0.5 μl | template |

The PCR protocol was as follows:

| | | |
|---|---|---|
| 94° C. | 2 | min |
| 98° C. | 10 | sec * |
| 55° C. | 30 | sec * |
| 68° C. | 30 | sec * |
| 68° C. | 2 | min |

* indicating 35 cycles, and finally cooled at 4° C. for use.

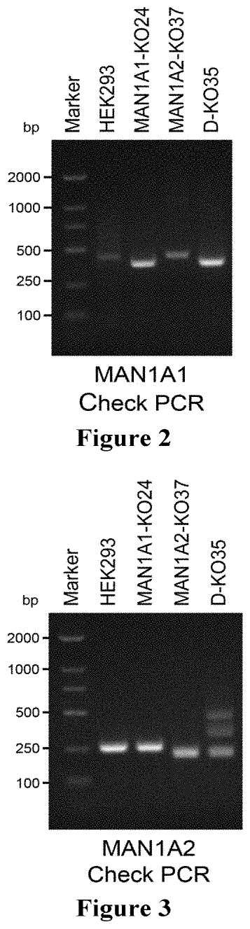

The results of the verification agarose gel electrophoresis were shown in FIGS. 2 and 3. In FIG. 2, the wild type WT and the single knocked-out cell strains MAN1A1KO24, MAN1A2KO37, and the double knocked-out cell strain MAN1A1/MAN1A2 DKO35 were compared. It can be seen that the band size was changed significantly from 431 bp before knocking out MAN1A1 to 358 bp after knocking out MAN1A1. Similarly, the band in FIG. 3 was also changed from the original 247 bp to 215 bp. SEQ ID NOs. 25 and 26 showed the primers for PCR-checking the MAN1A1 gene, and SEQ ID NOs. 27 and 28 showed the primers for PCR-checking the MAN1A2 gene.

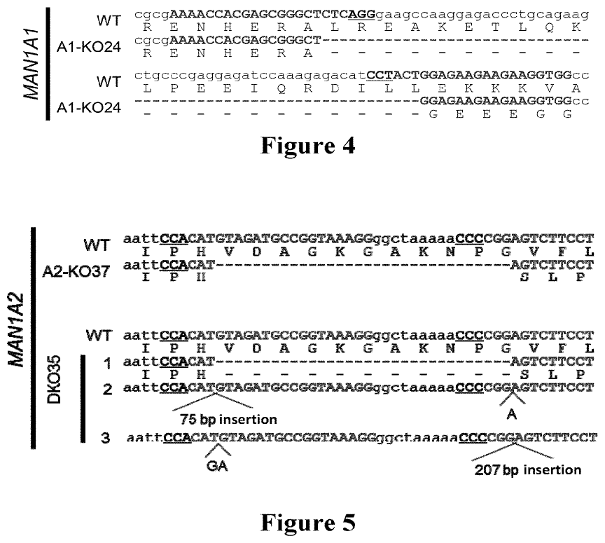

The knockout of the gene can be initially confirmed by comparing the change in the size of the band, SEQ ID NO: 33 showing the gene sequence of the wild type MAN1A1, SEQ ID NO: 34 showing the gene sequence of the single knocked-out cell strain MAN1A1KO24, SEQ ID NO: 35 showing the gene sequence of the wild type MAN1A2, and SEQ ID NO: 36 showing the gene sequence of single knockout of MAN1A2KO37. At the same time, after sequencing, the double knocked-out cell strain MAN1A1/MAN1A2 DKO35 was found to have multiple bands, and the sequencing results of the bands were analyzed and concluded that the Amp fragment inserted into the pX330-EGFP plasmid was in the band (see results in FIGS. 4 and 5, SEQ ID NOs: 37 and 38).

### EXAMPLE 2

Analysis of Sugar Chains on the Cell Surface by Flow Cytometry

After knocking out two genes MAN1A1/MAN1A2 encoding the alpha-mannosidases in the Golgi apparatus using the CRISPR/Cas9 system, the sugar chains on the surface of the double knocked-out cell strain will change to some extent. This phenomenon was confirmed by two different fluorescently labeled lectins. The lectin PHA-L4-FITC can recognize a complex sugar chain on the cell surface, and the lectin ConA-FITC can recognize a high-mannose type sugar chain on the cell surface. The types of sugar chains on the surface of different cell strains can be compared by staining the cells with lectin. The method was carried out as follows:

(1) inoculating different cell strains in a 6-well plate and allowing them to grow to 100%;

(2) removing the medium and rinsing the wells once with 1 ml of PBS;

(3) adding 220 μl of Tryp/EDTA to digest the cells;

(4) adding 1 ml of fresh 10% FCS medium to harvest the cells;

(5) centrifuging the cell solution at 3000 rpm for 3 min;

(6) resuspending in 1 ml of PBS and centrifuging again at the same rate, and repeating this step twice;

(7) adding 50 μl of 1% lectin solution (1% lectin+FACS solution) to the obtained cells and reacting for 15 min;

(8) adding 150 μl of FACS solution and centrifuging at 3000 rpm for 3 min;

(9) removing the supernatant after centrifugation;

(10) resuspending the cells by adding 200 μl of FACS solution again, then repeating centrifugation at 3000 rpm for 3 min;

(11) repeating steps (9) and (10) twice; and

(12) assaying the obtained sample by flow cytometry.

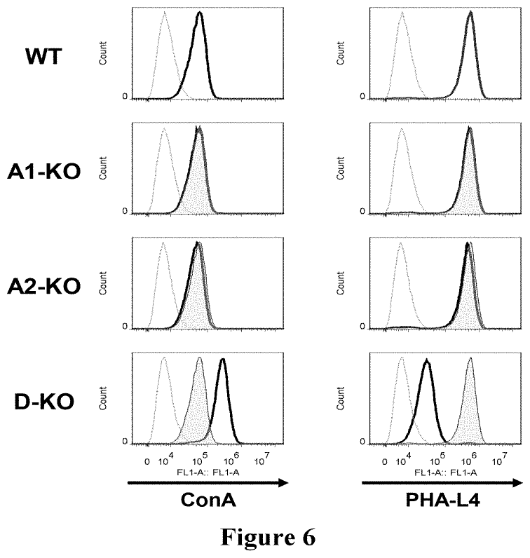

The result was shown in FIG. 6, and it can be seen that the amount of the complex sugar chains in the double knocked-out cell strain was significantly decreased compared with the wild type, while the proportion of the high-mannose type sugar chain was increased, but there was no significant change in the single knocked-out cell strains MAN1A1KO24 and MAN1A2KO37 as compared to the WT.

Preparation of FACS Solution:

| | | |
|---|---|---|
| PBS | 500 | ml |
| Albumin, Bovine, Frac-V | 5 | g |
| NaN3 | 0.5 | g |

### EXAMPLE 3

### Knocking Out Other Genes Related to the Alpha1,2-Mannosidase

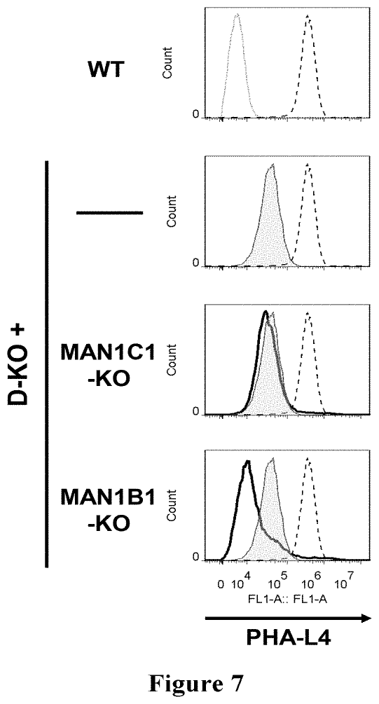

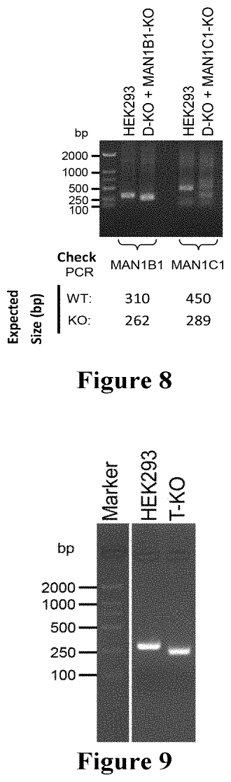

The plasmid for knocking out two genes MAN1C1 and MAN1B1 was introduced into the DKO cells (two knockout target sequences of the MAN1C1 gene and the corresponding primer sequences were set forth in SEQ ID NOs: 13 to 18, respectively, and two knockout target sequences of the MAN1B1 gene and the corresponding primer sequences were set forth in SEQ ID NOs: 19 to 24, respectively), and the plasmid introduced into the cell will express the sequences of the Cas9 protein and the target RNAs. The cells were cultured for about ten days after transfection, and the cell genome was extracted. Since two target sequence sites were designed, the gene sequence on the chromosome was displaced after the gene was knocked out, and the inventors confirmed the knockout of part of the genes (the results were shown in FIG. 8, and the SEQ ID NOs: 29 and 30 represent the primers for the PCR Check of the gene MAN1C1, and SEQ ID NOs: 31 and 32 represent the primers for the PCR check of the gene MAN1B1), and the sugar chains on the cell surface were analyzed using lectin ConA and PHA-L4 staining (see FIG. 7). After knocking out MAN1C1 from DKO cells, the sugar chain phenotype did not change, and after knocking out MAN1B1, the complex sugar chains were further reduced, proving that the gene was involved in the modification of sugar chain to form a complex sugar chain.

Construction of triple knocked-out cell strains of the genes MAN1A1, A2 and B1

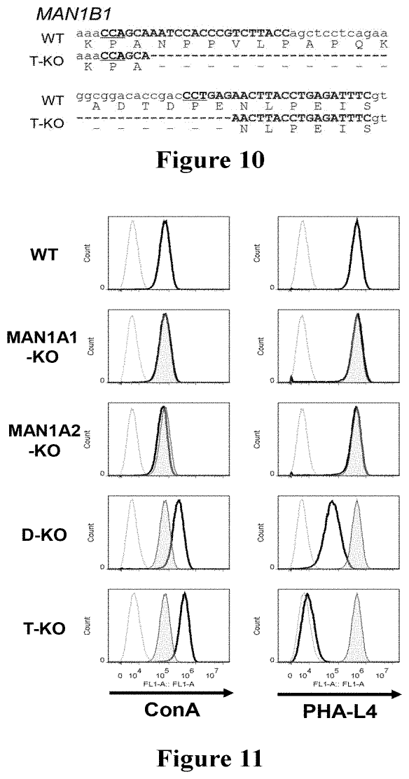

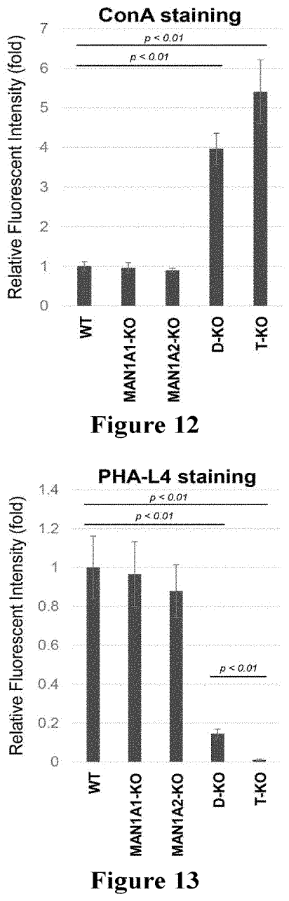

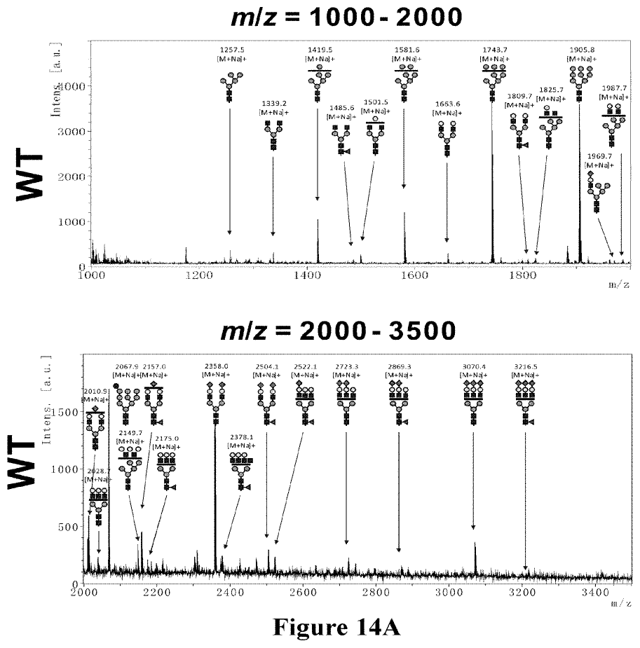

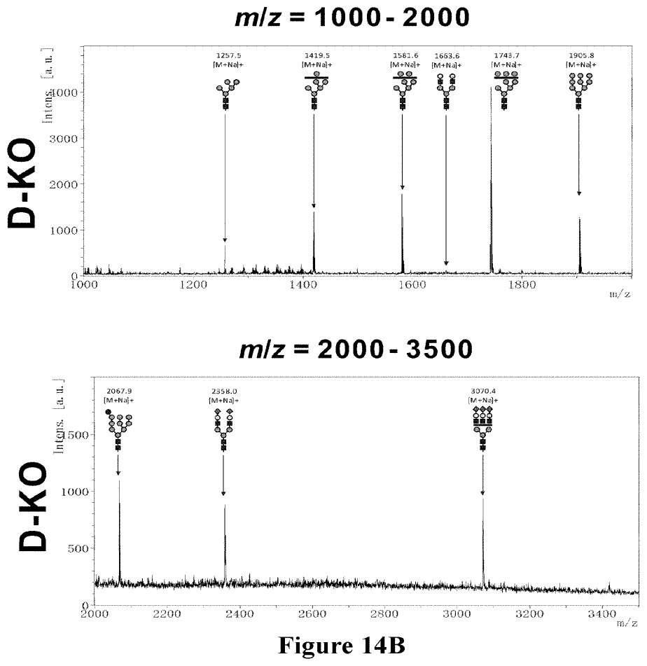

Since the cell sugar chain phenotype was further changed after knocking out MAN1B1, we further analyzed the cell strain. The DKO cell strain in which MAN1B1 was knocked out was named as TKO cells. There was a deletion of a 48 bp in size in the MAN1B1 coding sequence of the TKO cells (see FIGS. 9 and 10). SEQ ID NOs: 31 and 32 represent the primers for the PCR check of the MAN1B1 gene. Compared to wild-type and DKO cells, ConA staining showed a further increase in its high mannose-type sugar chains, while PHA-L4 staining showed almost complete attenuation of its signal (see FIG. 11). The inventors showed relative deviations in sugar chain phenotype changes between WT, single knocked-out cells MAN1A1KO24, MAN1A2KO37, DKO and TKO cells by relative fluorescence intensity after staining with lectin ConA-FITC and PHA-L4-FITC. The relative fluorescence intensity is to compare the mean value of the fluorescence intensity in the lectin staining results of the individual cells; setting the fluorescence intensity of the WT cells as the standard intensity of 1, the changes in the fluorescence intensity of each of the cell strains were compared. In the relative fluorescence intensity of ConA-FITC (see FIG. 12), there was no significant change in the relative intensity of the single knocked-out cell strains but there was a significant increase in the fluorescence intensity in the DKO and TKO cells. In contrast, in the relative fluorescence intensity of PHA-L4-FITC (see FIG. 13), there was a significant decrease in the fluorescence intensity of the DKO and TKO cells, with the relative value of the TKO cells being almost zero. In the figure, p<0.01 refers to the result of the P-value operation.

SEQ ID NO: 39 represents the gene sequence of the wild type MAN1B1, and SEQ ID NO: 40 represents the gene sequence of the cell strain MAN1A1/MAN1A2&B1 TKO.

### EXAMPLE 4

### Structural Analysis of Cellular Sugar Chains by MALDI-TOF

After further confirmation of the cell sugar chain type, it is necessary to structurally analyze the sugar chain. We used MALDI-TOF to determine the whole sugar chain of the cell.

1. Determination of Protein Concentration by BCA Kit

(1) Preparation of the Protein Standard Solution

30 mg of bovine serum albumin (BSA) was dissolved in 1.2 ml of water to form the protein standard initial solution at the concentration of 25 mg/ml. Then a series of protein standard solutions from 0.05 mg/ml were prepared with the protein standard initial solution as the mother solution (see the table below), to be placed at −20° C. for subsequent use.

| FINAL | | |
|---|---|---|
| CONCENTRATION | STANDARD | WATER |
| (MG/ML) | SAMPLE (UL) | (UL) |
| 0.5 | 24 μl of 25 mg/ml standard sample | 1176 |
| 0.4 | 560 μl of 0.5 mg/ml standard sample | 140 |
| 0.3 | 240 μl of 0.5 mg/ml standard sample | 160 |
| 0.2 | 300 μl of 0.4 mg/ml standard sample | 300 |
| 0.1 | 200 μl of 0.2 mg/ml standard sample | 1200 |
| 0 | 0 | 400 |

(2) Preparation of Working Solution

Each sample required 200 μl of the working solution, and the volume of the working solution required was calculated based on the number of samples and the standard solution. The reagents A and B were mixed in a ratio of 50:1, and ready to use.

(3) Determination of the Protein Concentration

a. Taking 20 μl of the standard and the samples to a 96-well plate;

b. Adding 200 μl of working solution to each well, mixing evenly and placing at 37° C. for 20-30 min, during which a microplate reader was preheated;

c. Measuring the absorbance of the sample at 562 nm;

d. Platting a standard curve using Excel and calculating the protein concentration.

2. Acetylhydrazine Modification and the Release of Sugar Chain Sialic Acid in the Protein Samples

After a collection tube for collecting waste liquid during ultrafiltration was mounted by a 10 kD ultrafiltration membrane, the sample protein solution in a volume corresponding to 1 mg of the protein was added, and each tube was filled with 8 mol/L urea to the same liquid surface, and mixed evenly. The solution was centrifuged at 14000 g for 15 min, and concentrated to the bottom of the ultrafiltration membrane, and the effluent was discarded. 300 μL of 8 mol/L urea was added, and the solution was centrifuged at 14,000 g for 15 min. 200 μL of 8 mol/L urea was further added, and the solution was centrifuged and the effluent was discarded. 150 μL of 10 mmol/L DTT solution was added and mixed evenly, and the solution was incubated at 56° C. for 45 min in a dry thermostat. After the reaction, the solution was centrifuged at 14000 g for 15 min in a benchtop centrifuge, and the effluent was discarded. 150 μL of 20 mmol/L IAM solution was added, pipetted thoroughly and mixed evenly, and it should be noted that light-avoiding operation of IAM. After mixed evenly, the ultrafiltration tube was placed in the dark to stand for 20 min. After the reaction, the solution was centrifuged at 14000 g for 15 min, and the effluent was discarded. 150 μL of ultrapure water was added and mixed evenly, the solution was centrifuged at 14000 g for 15 min, this step was repeated three times to wash away the IAM from the solution to avoid affecting the subsequent reaction. After washing, 100 μL of 1 mol/L acetohydrazide, 20 μL of 1 mol/L hydrochloric acid, and 20 μL of 2 mmol/L EDC were added, pipetted thoroughly and mixed evenly. The ultrafiltration tube was placed on a shaker of 120 rpm to ensure the protein to be suspended and reacted at room temperature for 4 hours. After the reaction, the solution was centrifuged at 14000 g in a tabletop centrifuge for 15 min, and the effluent was discarded. 150 μL of 40 mmol/L NH₄HCO₃ solution was added, pipetted thoroughly and mixed evenly, and centrifuged at 14000 g for 15 min, and washed with the NH₄HCO₃ solution for 3 times to provide a liquid phase environment of NH₄HCO₃ solution. The ultrafiltration tube was taken out and transferred to a clean collection tube, and 1 μL of PNGase-F dissolved in 300 μL of 40 mmol/L NH₄HCO₃ solution was added, pipetted thoroughly and mixed evenly, and placed in a thermostat incubator at 37° C. for 10-12 hours to enzymatically cleave the N-linked sugar chains. After the enzyme cleavage, the solution was centrifuged at 14000 g for 15 min, and the effluent was recovered. 150 μL of ultrapure water was added to the ultrafiltration membrane to pipette and resuspend the precipitated protein, and the solution was centrifuged at 15000 g for 15 min, repeating twice, and the N-linked sugar chains was completely collected, and the effluent was recovered in the collection tube. The ultrafiltration membrane was taken out and freeze-dried on a centrifugal concentrator to precipitate a sugar chain sample.

3. Clean Up Treatment of the Primary N-Linked Sugar Chain Samples

(1) Wash of Sepharose 4B:

In a 1.5 mL enzyme-free centrifuge tube, 100 μL of Sepharose 4B and 1mL of 1:1 methanol:water (V/V) solution were added and mixed evenly, centrifuged at 9000 g for 5 min, and left standstill vertically after centrifugation for 30 seconds. After the gel plane reached level, the supernatant was carefully extracted with a pipette and discarded, and the leftover was washed with methanol aqueous solution repeatedly for 5 times. 1 mL of n-butanol:methanol:water solution in a ratio of 5:1:1 (V/V) was added and mixed evenly, centrifuged at 9000 g for 5 min, and the supernatant was removed, and washed repeatly for 3 times to obtain the pretreated Sepharose 4B gel.

4. Loading the Sample and Cleaning Up to Purify N-Linked Sugar Chains:

500 μL of n-butanol:methanol:water solution at a ratio of 5:1:1 (V/V) was added to the freeze-dried and concentrated sugar chain sample to dissolve the sugar chain sample concentrated and crystallized at the bottom of the tube. After completely dissolved, the solution was loaded to the pretreated Sepharose 4B gel, mixed thoroughly, and reacted shakenly at a 80 r/min shaker for 1 h at room temperature. After the reaction, the solution was centrifuged at 9000 g in a tabletop centrifuge for 5 min, the supernatant was carefully pipetted and discarded, the leftover was washed with 700 μL of n-butanol:methanol:water solution at a ratio of 5:1:1 (V/V) repeatedly for 3 times; after washing, 500 μL of 1:1(V/V) methanol:water solution was added and mixed evenly, reacted at a 140 r/min shaker for 20 min at room temperature, and the N-linked sugar chains bound to Sepharose 4B gel were eluted. After the reaction, the solution was centrifuged at 9000 g for 5 min, and the supernatant was collected in a new 1.5 mL enzyme-free centrifuge tube. The elution was repeated once, and the collected sugar chain sample solution was freeze-dried on a centrifugal concentrator to precipitate a Cleaned-up sugar chain sample.

5. Data Analysis

The sugar chain mass spectrometry data were shown in the flexAnalysis software, and the signal-to-noise ratio was greater than 5, and the mass spectrum peaks identified in at least three experiments were subjected to subsequent analysis.

The m/z and signal intensity results of the resultant sugar chains were exported to a file in txt format.

The sugar chain structure was manually analyzed in combination with Glycoworkbench software. The analysis parameters were: GlycomeDB database, ion [M+Na]+, charge up to +1, precursor ion tolerance of 1 Da, and fragmentation ion tolerance of 0.5 Da.

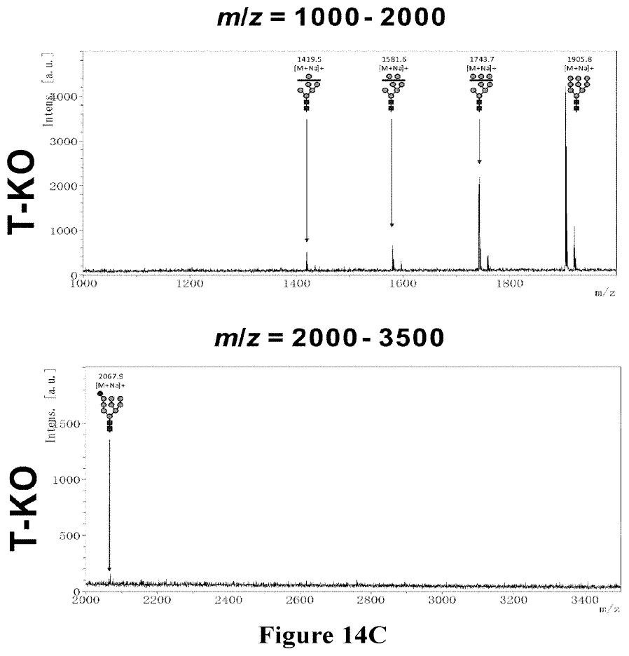

FIG. 14 showed the total sugar chain comparison of the wild type cell WT, double knocked-out cell strain DKO and triple knocked-out cell strain TKO. It can be seen that the diversity of sugar chains in the double knockout cell strain was reduced, but a complex sugar chain structure was still present. However, only knocking out the Golgi mannosidase I gene did not make the sugar chain structures to be substantially homogeneous. The sugar chains of the triple knocked-out cell strain were more homogeneous, and the main sugar chain structure was high-mannose type N-linked sugar chains.

6. Solution Preparation:

40 mmol/L of NH₄HCO₃: 0.0316 g of NH₄HCO₃ was weighed and dissolved in 10 ml of ultrapure water;

10 mmol/L of DTT: 0.0154 g of DL-Dithiothreitol was weighed and dissolved in 1 ml of 40 mmol/L NH₄HCO₃ to make a 10× mother solution which was diluted 10 times into a working solution;

20 mmol/L of IAM: 0.037 g of Iodoacetamide was weighed and dissolved in 1 ml of 40 mmol/L NH₄HCO₃ to prepare a 10× mother solution which was diluted as a working solution. (Kept in dark);

1 mol/L of acetohydrazide: 0.074 g of acetohydrazide was weighed and dissolved in 1 ml of ultrapure water;

2 mol/L of EDC: 0.0383 g of EDC was weighed and dissolved in 100 ml of ultrapure water;

1 mol/L of hydrochloric acid: 100 μI of 37% concentrated hydrochloric acid was weighed and dissolved in 1.10 ml of ultrapure water;

8 mol/L of urea: 4.83032 g of urea was weighed and dissolved with ultrapure water to a final volume of 10 ml.

The inventors analyzed the sugar chains of the DKO and TKO cells. Total cell proteins were extracted from the WT, DKO and TKO cells. The sialic acid on the N-linked sugar chains was amidated and the sugar chains were released from the protein by treatment with PNGaseF. The amidated N-linked sugar chains were then subjected to MALDI-TOF analysis (the results can be seen in FIG. 14). There were at least 27 different types of sugar chains in the WT cells, including high-mannose, hybrid and complex type sugar chains (FIG. 14A). The complex sugar chain has diantennary and triantennary structures, and there were also unsialylated and fucosylated sugar chains. On the other hand, the diversity of sugar chains in the DKO cells was reduced and the high-mannose type sugar chain was the main type, but the complex type sugar chain was still present (FIG. 14B), but the complex type sugar chains were simplified to sialylated diantennary, disialylated diantennary and triantennary sugar chain structures. The Man8GlcNAc2 structure in the DKO cells was the main sugar chain structure; in the TKO cells, the sugar chain structure was further simplified meanwhile the complex type sugar chains were below the detectable limit (FIG. 14C), and all the detectable sugar chain structures were the high mannose type. Compared with the WT cells, Man9GlcNAc2 and Man8GlcNAc2 were the main structures in the DKO and TKO cells. These results were consistent with lectin staining results, indicating significant changes of the sugar chain structure in the DKO and TKO cells, and the high mannose type sugar chains were significantly increased.

### EXAMPLE 5

### Analysis of Sugar Chain Changes and Type Discrimination by Western Blotting

In order to construct the pME-pgkpuro-sHF-GLA and pME-pgkepuro-sHF-LIPA plasmids, the DNA fragments encoding mature alpha-galactosidase A (GLA) and mature lysosomal lipase (LIPA) were enriched by PCR, and linked to the pME-puro plasmids with XhoI and NotI sites. The plasmids carried an ER signal sequence CD59 and a His6-Flag sequence.

Transfection Method:

The wild type cells HEK293, DKO and TKO cells were cultured overnight with a medium containing 10% FCS and used for transfection when they grown to be approximately 90-95% confluent. PEI-MAX (2 mg/ml PH 7.5) was used as the transfection reagent, and the PEI-MAX and OPTI (life technologies: 31985-070) were mixed evenly at a ratio of 1 μI PEI-MAX: 50 μl OPTI medium before transfection. The plasmids for knocking out and the plasmid pME-puro carrying the resistance gene were mixed evenly with the OPTI medium, and the amount of the plasmid added was: 4 μg DNA: 5 μl PEI-MAX. The PEI-MAX solution was mixed with the plasmid-containing solution and allowed to stand at room temperature for 25 minutes to let the plasmid bind to PEI-MAX. The mixed solution was then added to the medium of the wild-type cell strain. The medium was replaced with the fresh medium every 12 hours, and after the growth was resumed (about 24 hours), the medium was changed to a medium containing puromycin at a concentration of 1 μg/ml for screening.

1. Sample Preparation

(1) inoculating 5*10⁵ cells in 6-well plates and culturing for 12 hours

(2) replacing the medium with new medium containing 10% FCS and culturing for another 48 hours

(3) collecting the cells and the culture medium

a. the cells

(1) removing the medium and rinsing with PBS

(2) harvesting the cells with tryp/EDTA

(3) transferring the cell solution to an EP tube, centrifuging at 3000 rpm at 3° C. for 3 min

(4) removing the supernatant and adding 100 μI of the cell lysate

(5) placing on ice for 30 min

(6) centrifuging at 10000 g at 4° C. for 15 min

(7) taking 90 μI of the supernatant to a new EP tube and adding 30 μI of 4× sample buffer

(8) boiling at 95° C. for 5 min

b. the medium

(1) collecting 1.4 ml of the medium

(2) centrifuging at 10000 g at 4° C. for 5 min

(3) transferring 1 ml of the supernatant to a new EP tube

(4) adding 20 μI of anti-Flag beads (washing three times with PBS)

(5) reacting shakenly at 4° C. for 2 hours

(6) centrifuging at 10000 g at 1° C. for 4 min

(7) removing the supernatant

(8) adding 1 ml of PBS

(9) repeating steps 6-8 at least three times

(10) adding 50 μI of elution buffer containing Flag-peptide

(11) reacting shakenly at 4° C. for 2 hours

(12) taking 45 μI of the supernatant into a new EP tube

(13) adding 15 μI of 4× sample buffer

(14) boiling the protein at 95° C. for 5 min

2. Enzyme Cleavage Reaction

(1) PNGaseF Reaction

| | TPTAL = 20 ML |
|---|---|
| PNGASEF | 1 UL |
| SAMPLE | 10 UL |
| DD WATER | 5 UL |
| 10% NP40 | 2 UL |
| GLYCOBUFFER2 | 2 UL |

### Reacting for 3 h

(2) EndoH Reaction

| | TOTAL = 20 ML |
|---|---|
| ENDOH | 1 UL |
| DDWATER | 7 UL |
| GLYCOBUFFER3 | 2 UL |
| SAMPLE | 10 UL |

Reacting for 3 h

3. Western Blotting

(1) placing filter paper, PVDF membrane, gel, and filter paper in the order of from top to bottom in the electrophoresis instrument

(2) conducting transmembrane at 25V 1.0 A for 30 min

(3) washing the membrane with the TBST buffer three times

(4) blocking with 5% skim milk for 1 h

(5) incubating with the 4000× diluted primary antibody (anti-Flag Mouse mAb) in milk for 3 h at room temperature

(6) washing with the TBST for 30 min

(7) adding the 4000× diluted secondary antibody (goat Anti-Mouse IgG, HRP) and incubating for 1 h

(8) washing with the TBST for 30 min

(9) developing using ECL chromogenic reagent (BIO-RAD) and placing in the ImageQuant LAS 4000 gel imaging system to visualize.

SEQ ID NO: 41 represents the DNA sequence for expressing alpha-lysosomal lipase to be inserted into the expression vector, and SEQ ID NO: 42 represents the DNA sequence for expressing alpha-lysosomal galactosidase to be inserted into the expression vector.

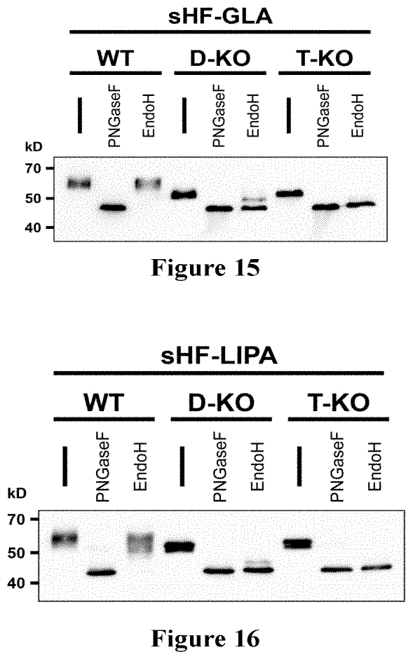

FIG. 15 showed the comparison results of the wild-type cells, double knocked-out cell strains and triple knocked-out cell strains with His-Flag tagged alpha-galactosidase A (GLA), since the sugar chains cannot be cleaved by EndoH, it can be concluded that the surface of the alpha-galactosidase of the wild-type cell was mainly the complex type sugar chains. The sugar chains of the double knocked-out cell strains can be cleaved by EndoH or partially by PNGaseF, proving that the alpha-galactosidase A sugar chain was mainly composed of high mannose type sugar chain. Although some sugar chains were still slightly heterogeneous, i.e. the high mannose type sugar chain is not the only type in the protein expressed in the double knocked-out cells, some non-high mannose type sugar chains were still present. However, the proportion of high mannose-type sugar chains in the total sugar chains was greatly increased relative to the wild-type cells. In the triple knocked-out cell strains, the sugar chain can be cleaved by EndoH and PNGaseF, proving that the alpha-galactosidase sugar chain was mainly composed of high mannose type sugar chains, and also proving the homogeneity of sugar chains in the triple knocked-out cell strains. In the same way, the same expression experiment was performed on the lysosomal lipase (LIPA), and the results were consistent with the above results. The results are shown in FIG. 16.

In addition, it can be seen from bands obtained from the endoH sensitivity experiment, i.e. the reaction of the secreted alpha-galactosidase A (GLA) recombinant protein with EndoH in the western blot, that the ratio of the sugar chain of the protein in the alpha-galactosidase A (GLA) protein secreted by the wild-type (WT) cells which was high-mannose-type sugar chain was 0.05%; the ratio of the sugar chain of the protein in the alpha-galactosidase A (GLA) protein secreted by the DKO cells which was the high mannose type sugar chain was 82.35%; and the ratio of the sugar chain of the protein in the alpha-galactosidase A (GLA) protein secreted by the TKO cells which was the high mannose type sugar chain was 97.5%. Similarly, EndoH was used to treat the lysosomal lipase (LIPA), and the ratio of the sugar chain of the protein in the lysosomal lipase (LIPA) protein secreted by the wild type (WT) cells which was the high mannose type sugar chain was 0.26%; the ratio of the sugar chain of the protein in the lysosomal lipase (LIPA) protein secreted by the DKO cells which was the high mannose type sugar chain was 81.23%, and the ratio of the sugar chain of the protein in the lysosomal lipase (LIPA) protein secreted by the TKO cells which was the high mannose type sugar chain was 99.14%.

Thus, it can be seen that, according to the present invention, the homogeneity of the sugar chains in the glycoprotein was greatly increased, and the ratio of the high mannose type sugar chain was increased to more than 80%, and even more than 99%.

### EXAMPLE 6

### Analysis of Sugar Chain Structure on Proteins

To express the sHF-LIPA protein in the wild-type cells and the T-KO cell strain, the expression plasmid pHEK293Ultra-sHF-LIPA was first transfected into the cell strain (3 culture dishes of 15 cm). The next day, the medium was changed and the cells were cultured for further 3 days. After 3 days, 75 ml of the medium was collected, and the secreted sHF-LIPA protein was purified by 750 μl Ni-NTA agarose and eluted using an elution buffer (250 mM imidazole solution, pH 7.4). The eluted sHF-LIPA solution was further purified using 40 μl anti-Flag beads (SIGMA). The protein bound to anti-Flag beads was eluted by 300 μl of Flag peptide solution (500 μg/ml).

In order to express EGFP-F-IgG1 in the wild-type cells and the T-KO cell strain, the wild-type and a T-KO cell strain stably expressing EGFP-F-HyHEL10 (EGFP-F-IgG1) were constructed by transfection with retroviral vectors pLIB2-pgkHyg-ssEGFP-F-HyHEL10 and pLIB2-pgkBSD-HyHEL10-human-kappa. After the cells (in 10 culture dishes of 15 cm) were cultured for 3 days, 250 ml of the medium was collected, and the EGFP-F-IgG1 protein was purified using protein-A Sefinose resin, and further purified using 40 μl of anti-Flag beads. The purified EGFP-F-IgG1 was confirmed by Coomassie Brilliant Blue (CBB) staining.

For sugar chain analysis, the purified sHF-LIPA protein was separated in SDS-PAGE by electrophoresis, and then transferred to a PVDF membrane. The PVDF membrane was stained with Direct Blue-71 (SIGMA) and the Direct Blue-71 did not interfere with the MALDI-TOF mass spectrometry signal. The stained sHF-LIPA band was excised from the membrane and transferred to a microtube. After soaking the membrane in the microtube with methanol, the methanol was removed, and the PVDF membrane was blocked with polyvinyl alcohol (PVA). After removing the PVA, 30 μl of 50 mM ammonium bicarbonate solution containing 2 mU PNGase F (TAKARA) was added (pH 7.8), then incubated for 18 hours at 37 degrees Celsius. For sugar chain analysis of the sugar chains on the EGFP-F-IgG1 protein, the N-linked sugar chains were released from the purified EGFP-F-IgG1 using PNGase F. The samples obtained in the microtube were purified using the BlotGlyco sugar chain purification kit according to the instructions (Sumitomo Bakelite). In brief, the sugar chains released in the solution were captured by BlotGlyco beads, followed by methylation of the sialic acids on the sugar chains using 3-methyl-1-p-tolytriazene (SIGMA), and the captured sugar chains were labeled and released using aminooxy-functionalized peptide reagent (aoWR). The labeled sugar chains were eluted in a resin column using 50 μl of deionized water. Finally, elution was performed using the purification column provided in the kit to obtain a sugar chain-containing solution for mass spectrometry.

Mass spectrometry was performed using MALDI/TOF-MS (Bruker Daltonics). The ions were excited using a pulsed 337 nm nitrogen laser and accelerated to 25 kV. Mass spectral data were obtained using a reflector mode with 200 ns delayed extraction. For sample preparation for mass spectrometry, 0.5 μl of 30% ethanol DHB (10 mg/ml) solution was spotted onto the target plate (MTP 384 target plate ground steel, Bruker) and air-dried, and then 0.5 μl of the sugar chain sample was spotted on DHB crystals and air-dried.

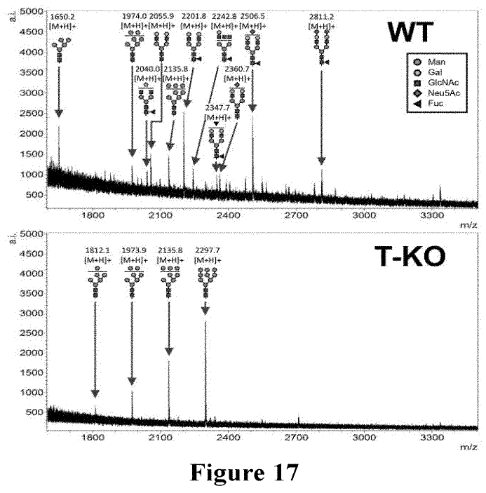

FIG. 17 showed the results of the purified LIPA in the wild type, showing there were more than 30 N-linked sugar chain structure forms, and high mannose type, hybrid type and complex type sugar chains were present. In particular, there were a large number of fucosylated and sialylated structures in the sugar chain structures. In contrast, the sugar chains on the LIPA protein expressed from the T-KO cell strain was more simplified, and the main sugar chain structure was high mannose type, however, there were also some peaks of complex sugar chains in the results.

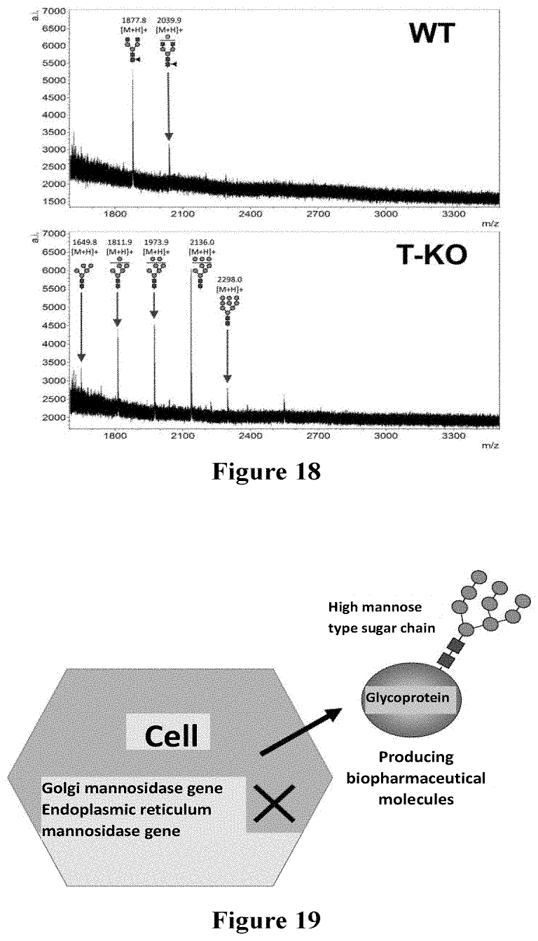

FIG. 18 further showed the sugar chain structure of the EGFP-Flag-labeled human IgG1 expressed in the wild-type and the T-KO cells. In the wild-type cell strain, there were several fucosylated diantennary complex sugar chain structures. On the other hand, in the results of the IgG1 expressed in the T-KO cells, most of the sugar chain structures were converted to high mannose type. These data indicated that the N-linked sugar chain structure was simplified on the secreted proteins, converting from complex sugar chains to high mannose type sugar chains at the protein level.

The cell strains of the present application were described using gene knocked-out cell strains as examples, but it is obvious that the inventive concept of the present application is not limited to the above cell strains and the specific lysosomal hydrolases produced therefrom. It is clear to those skilled in the art that the present invention also applies to the production of other glycoproteins and to other lysosomal storage diseases.

Each sequence in the sequence listing represents:

SEQ ID NO: 1: MAN1A1-KO Target Sequence 1

SEQ ID NO: 2: MAN1A1-KO Primer KO1F

SEQ ID NO: 3: MAN1A1-KO Primer KO1R

SEQ ID NO: 4: MAN1A1-KO Target Sequence 2

SEQ ID NO: 5: MAN1A1-KO Primer KO2F

SEQ ID NO: 6: MAN1A1-KO Primer KO2R

SEQ ID NO: 7: MAN1A2-KO Target Sequence 1

SEQ ID NO: 8: MAN1A2-KO Primer KO1F

SEQ ID NO: 9: MAN1A2-KO Primer KO1R

SEQ ID NO: 10: MAN1A2-KO Target Sequence 2

SEQ ID NO: 11: MAN1A2-KO Primer KO2F

SEQ ID NO: 12: MAN1A2-KO Primer KO2R

SEQ ID NO: 13: MAN1C1-KO Target Sequence 1

SEQ ID NO: 14: MAN1C1-KO Primer KO1F

SEQ ID NO: 15: MAN1C1-KO Primer KO1R

SEQ ID NO: 16: MAN1C1-KO Target Sequence 2

SEQ ID NO: 17: MAN1C1-KO Primer KO2F

SEQ ID NO: 18: MAN1C1-KO Primer KO2R

SEQ ID NO: 19: MAN1B1-KO Target Sequence 1

SEQ ID NO: 20: MAN1B1-KO primer KO1F

SEQ ID NO: 21: MAN1B1-KO Primer KO1R

SEQ ID NO: 22: MAN1B1-KO Target Sequence 2

SEQ ID NO: 23: MAN1B1-KO Primer KO2F

SEQ ID NO: 24: MAN1B1-KO Primer KO2R

SEQ ID NO: 25: MAN1A1-Test Primer F

SEQ ID NO: 26: MAN1A1-Test Primer R

SEQ ID NO: 27: MAN1A2-Test Primer F

SEQ ID NO: 28: MAN1A2-Test Primer R

SEQ ID NO: 29: MAN1C1-Test Primer F

SEQ ID NO: 30: MAN1C1-Test Primer R

SEQ ID NO: 31: MAN1B1-Test Primer F

SEQ ID NO: 32: MAN1B1-Test Primer R

SEQ ID NO: 33: the wild-type WT gene sequence in the experiment for verifying the knockout of the gene MAN1A1

SEQ ID NO: 34: the gene sequence of the MAN1A1 knockout cell strain MAN1A1KO24 in the experiment for verifying the knockout of the gene MAN1A1

SEQ ID NO: 35: the wild-type WT gene sequence in the experiment for verifying the knockout of the gene MAN1A2

SEQ ID NO: 36: the gene sequence of the MAN1A2 knockout cell strain MAN1A2KO37 in the experiment for verifying the knockout of the gene MAN1A2

SEQ ID NO: 37: the gene sequence type1 of double knocked-out cell strain MAN1A1/A2 DMKO35 in the gene MAN1A2 knockout experiment

SEQ ID NO: 38: the gene sequence type2 of double knocked-out cell strain MAN1A1/A2 DMKO35 in the gene MAN1A2 knockout experiment

SEQ ID NO: 39: the wild-type WT gene sequence in the experiment for verifying the knockout of the gene MAN1B1

SEQ ID NO: 40: the gene sequence of the triple knocked-out cell strain MAN1A1/A2&B1 TKO2 in the experiment for verifying the knockout of the gene MAN1B1

SEQ ID NO: 41: DNA sequence for expressing alpha-lysosomal lipase to be inserted in the expression vector

SEQ ID NO: 42: DNA sequence for expressing alpha-lysosomal galactosidase to be inserted in the expression vector

SEQ ID NO: 43: DNA sequence of human MAN1B1

SEQ ID NO: 44: DNA sequence of human MAN1A1

SEQ ID NO: 45: DNA sequence of human MAN1A2

SEQ ID NO: 46: DNA sequence of human MAN1C1

1. An animal cell strain capable of producing a glycoprotein having a high-mannose type sugar chain as a main N-linked sugar chain structure, wherein at least two genes of the Golgi mannosidase and endoplasmic reticulum mannosidase genes in the cell strain are destroyed or knocked out.

2. The animal cell strain according to claim 1, wherein the high-mannose type sugar chain is at least one selected from the group consisting of Glc1-Man9-GlcNAc2, Man9-GlcNAc2, Man8-GlcNAc2, Man7-GlcNAc2, Man6-GlcNAc2 and Man5-GlcNAc2.

3. The animal cell strain according to claim 1, wherein the cell strain is derived from a mammalian cell selected from the group consisting of human embryonic kidney cell (HEK293), Chinese hamster ovary cell (CHO), COS, 3T3, myeloma, BHK, HeLa and Vero, or an amphibian cell selected from the group consisting of Xenopus egg cells or an insect cell Sf9, Sf21 or Tn5.

4. The animal cell strain according to claim 3, wherein the cell strain is derived from human embryonic kidney cell (HEK293) or Chinese hamster ovary cell (CHO).

5. The animal cell strain according to claim 1, wherein
the destroying is achieved by a gene-destroying method targeting a Golgi mannosidase gene and/or an endoplasmic reticulum mannosidase gene, and/or
the knockout is achieved by a gene knockout method targeting a Golgi mannosidase gene and/or an endoplasmic reticulum mannosidase gene.

6. The animal cell strain according to claim 5, wherein the endoplasmic reticulum mannosidase is:
(a) a protein encoded by the DNA sequence as set forth in SEQ ID NO: 43, or
(b) a protein having more than 20% homology with the amino acid sequence of the protein encoded by the DNA sequence as set forth in SEQ ID NO: 43 and having endoplasmic reticulum mannosidase activity.

7. The animal cell strain according to claim 5, wherein the Golgi mannosidase is:
(a) a protein encoded by the DNA sequence as set forth in SEQ ID NO: 44,
(b) a protein having more than 20% homology with the amino acid sequence of the protein encoded by the DNA sequence as set forth in SEQ ID NO: 44 and having Golgi mannosidase I activity,
(c) a protein encoded by the DNA sequence as set forth in SEQ ID NO: 45,
(d) a protein having more than 20% homology with the amino acid sequence of the protein encoded by the DNA sequence as set forth in SEQ ID NO: 45 and having Golgi mannosidase I activity,
(e) a protein encoded by the DNA sequence as set forth in SEQ ID NO: 46, or
(f) a protein having more than 20% homology with the amino acid sequence of the protein encoded by the DNA sequence as set forth in SEQ ID NO: 46 and having Golgi mannosidase I activity.

8. The animal cell strain according to claim 1, wherein the Golgi mannosidase gene is selected from the group consisting of the Golgi mannosidase I genes MAN1A1, MAN1A2 and MAN1C1, and/or the endoplasmic reticulum mannosidase gene is the endoplasmic reticulum mannosidase gene MAN1B1.

9. The animal cell strain according to claim 1, wherein two genes selected from the group consisting of the Golgi mannosidase I genes MAN1A1, MAN1A2 and MANIC1 in the cell strain are knocked out.

10. The animal cell strain according to claim 9, wherein the cell strain is the cell strain A1/A2-double-KO with the genes MAN1A1/A2 double knocked-out (with the deposit number CTCCC No: C201767)

11. The animal cell strain according to claim 1, wherein three genes selected from the group consisting of the Golgi mannosidase I genes MAN1A1, MAN1A2 and MAN1C1 and the endoplasmic reticulum mannosidase gene MAN1B1 in the cell strain are knocked out.

12. The animal cell strain according to claim 11, wherein the cell strain is the cell strain A1/A2/B1-triple-KO with the genes MAN1A1/A2/B1 triple knocked-out (with the deposit number CTCCC No: C2016193).

13. The animal cell strain according to claim 1, wherein the glycoprotein is a lysosomal enzyme or an antibody.

14. The animal cell strain according to claim 13, wherein the lysosomal enzyme is human alpha-galactosidase or human lysosomal lipase.

15. A method for producing a glycoprotein having a high-mannose type sugar chain as a main N-linked sugar chain structure, the method comprising culturing the animal cell strain according to claim 1.

16. A glycoprotein having a high-mannose type sugar chain as a main N-linked sugar chain structure prepared by the method according to claim 15.

17. The glycoprotein according to claim 16, wherein the glycoprotein is human alpha-galactosidase or human lysosomal lipase.

18. A method of treating a lysosomal storage disease. comprising administering the glycoprotein of claim 16 to a subject in need thereof.

19. The method according to claim 18, wherein the lysosomal storage disease is Fabry's disease.

20. The method according to claim 18, wherein the lysosomal storage disease is Wolman's disease or cholesterol ester storage disease.


SEQUENCE LISTING (46 sequences)

SEQ ID NO: 1 - DNA, 23 bp, Homo sapiens
aaaaccacgagcgggctctcagg

SEQ ID NO: 2 - DNA, 24 bp, Artificial sequence
  MAN1A1-KO Primer KO1F
caccaaaaccacgagcgggctctc

SEQ ID NO: 3 - DNA, 24 bp, Artificial sequence
  MAN1A1-KO Primer KO1R
aaacgagagcccgctcgtggtttt

SEQ ID NO: 4 - DNA, 23 bp, Homo sapiens
ccaccttcttcttctccagtagg

SEQ ID NO: 5 - DNA, 24 bp, Artificial sequence
  MAN1A1-KO Primer KO2F
caccccaccttcttcttctccagt

SEQ ID NO: 6 - DNA, 24 bp, Artificial sequence
  MAN1A1-KO Primer KO2R
aaacactggagaagaagaaggtgg

SEQ ID NO: 7 - DNA, 23 bp, Homo sapiens
cctttaccggcatctacatgtgg

SEQ ID NO: 8 - DNA, 24 bp, Artificial sequence
  MAN1A2-KO Primer KO1F
cacccctttaccggcatctacatg

SEQ ID NO: 9 - DNA, 24 bp, Artificial sequence
  MAN1A2-KO Primer KO1R
aaaccatgtagatgccggtaaagg

SEQ ID NO: 10 - DNA, 23 bp, Homo sapiens
catggatcaggaagactccgggg

SEQ ID NO: 11 - DNA, 24 bp, Artificial sequence
  MAN1A2-KO Primer KO2F
cacccatggatcaggaagactccg

SEQ ID NO: 12 - DNA, 24 bp, Artificial sequence
  MAN1A2-KO Primer KO2R
aaaccggagtcttcctgatccatg

SEQ ID NO: 13 - DNA, 23 bp, Homo sapiens
ttgctgaatgtctctcccggggg

SEQ ID NO: 14 - DNA, 24 bp, Artificial sequence
  MAN1C1-KO Primer KO1F
caccttgctgaatgtctctcccgg

SEQ ID NO: 15 - DNA, 24 bp, Artificial sequence
  MAN1C1-KO Primer KO1R
aaacccgggagagacattcagcaa

SEQ ID NO: 16 - DNA, 23 bp, Homo sapiens
gtatgactcgtgacacgtcttgg

SEQ ID NO: 17 - DNA, 24 bp, Artificial sequence
  MAN1C1-KO Primer KO2F
caccgtatgactcgtgacacgtct

SEQ ID NO: 18 - DNA, 24 bp, Artificial sequence
  MAN1C1-KO Primer KO2R
aaacagacgtgtcacgagtcatac

SEQ ID NO: 19 - DNA, 23 bp, Homo sapiens
gcaaatccacccgtcttaccagc

SEQ ID NO: 20 - DNA, 24 bp, Artificial sequence
  MAN1B1-KO primer KO1F
caccgcaaatccacccgtcttacc

SEQ ID NO: 21 - DNA, 24 bp, Artificial sequence
  MAN1B1-KO Primer KO1R
aaacggtaagacgggtggatttgc

SEQ ID NO: 22 - DNA, 23 bp, Homo sapiens
gaaatctcaggtaagttctcagg

SEQ ID NO: 23 - DNA, 24 bp, Artificial sequence
  MAN1B1-KO Primer KO2F
caccgaaatctcaggtaagttctc

SEQ ID NO: 24 - DNA, 24 bp, Artificial sequence
  MAN1B1-KO Primer KO2R
aaacgagaacttacctgagatttc

SEQ ID NO: 25 - DNA, 21 bp, Artificial sequence
  MAN1A1-Test Primer F
ttcctgccagactcctccaag

SEQ ID NO: 26 - DNA, 24 bp, Artificial sequence
  MAN1A1-Test Primer R
ccactcacctctttgatctttgcc

SEQ ID NO: 27 - DNA, 24 bp, Artificial sequence
  MAN1A2-Test Primer F
cttagtgccttcatcactctgtgt

SEQ ID NO: 28 - DNA, 23 bp, Artificial sequence
  MAN1A2-Test Primer R
gacagattcgatccaatcaccgt

SEQ ID NO: 29 - DNA, 22 bp, Artificial sequence
  MAN1C1-Test Primer F
caggcatcttgaccagcagaga

SEQ ID NO: 30 - DNA, 24 bp, Artificial sequence
  MAN1C1-Test Primer R
ttccagttcttcctggtgtctagc

SEQ ID NO: 31 - DNA, 20 bp, Artificial sequence
  MAN1B1-Test Primer F
tctcttgtaggccttgtggc

SEQ ID NO: 32 - DNA, 22 bp, Artificial sequence
  MAN1B1-Test Primer R
gagagagaggtaaacacgtggt

SEQ ID NO: 33 - DNA, 440 bp, Homo sapiens
gcttcggggcgatcttcttcctgccagactcctccaagctgctcagcggggtcctgttccactccagccccgccttgcagccggccgccgaccacaagcccgggcccggggcgcgcgccgaggacgcggccgaggggcgagcccggcgccgcgaggagggggcacccggggacccggaggccgccctggaggacaacttggccaggatccgcgaaaaccacgagcgggctctcagggaagccaaggagaccctgcagaagctgcccgaggagatccaaagagacatcctactggagaagaagaaggtggcccaggaccagctgcgtgacaaggcgccgttcagaggcctgcccccggtggacttcgtgcccccaatcggggtggagagccgggagcccgccgacgccgccatccgcgagaaaagggcaaagatcaaagag

SEQ ID NO: 34 - DNA, 378 bp, unknown
  Gene sequence of the MAN1A1 knockout cell strain MAN1A1KO24
gcttcggggcgatcttcttcctgccagactcctccaagctgctcagcggggtcctgttccactccagccccgccttgcagccggccgccgaccacaagcccgggcccggggcgcgcgccgaggacgcggccgaggggcgagcccggcgccgcgaggagggggcacccggggacccggaggccgccctggaggacaacttggccaggatccgcgaaaaccacgagcgggctggagaagaagaaggtggcccaggaccagctgcgtgacaaggcgccgttcagaggcctgcccccggtggacttcgtgcccccaatcggggtggagagccgggagcccgccgacgccgccatccgcgagaaaagggcaaagatcaaagag

SEQ ID NO: 35 - DNA, 247 bp, Homo sapiens
cttagtgccttcatcactctgtgttttggggcattctttttccttccagactcttcaaaacacaaacgctttgatttgggtttagaagatgtgttaattccacatgtagatgccggtaaaggggctaaaaaccccggagtcttcctgatccatggacccgatgaacatagacacaggtttgtttatttcagaagttctggtactaacatttggcagagcaaggtacggtgattggatcgaatctgtc

SEQ ID NO: 36 - DNA, 215 bp, unknown
  Gene sequence of the MAN1A2 knockout cell strain MAN1A2KO37
cttagtgccttcatcactctgtgttttggggcattctttttccttccagactcttcaaaacacaaacgctttgatttgggtttagaagatgtgttaattccacatagtcttcctgatccatggacccgatgaacatagacacaggtttgtttatttcagaagttctggtactaacatttggcagagcaaggtacggtgattggatcgaatctgtc

SEQ ID NO: 37 - DNA, 218 bp, unknown
  Gene sequence type1 of double knocked-out cell strain MAN1A1/A2 DMKO35
cttagtgccttcatcactctgtgttttggggcattctttttccttccagactcttcaaaacacaaacgctttgatttgggtttagaagatgtgttaattccacatagtcttcctgatccatggacccgatgaacatagacacaggtttgtttatttcagaagttctggtactaacatttggcagagcaaggtacggtgattggatcgaatctgtcatc

SEQ ID NO: 38 - DNA, 325 bp, unknown
  Gene sequence type2 of double knocked-out cell strain MAN1A1/A2 DMKO35
gacttagtgccttcatcactctgtgttttggggcattctttttccttccagactcttcaaaacacaaacgctttgatttgggtttagaagatgtgttaattccacattggtatggcttcattcagctccggttcccaacgatcaaggcgagttacatgatcccccatgttgtgcaaaaaagcgtagatgccggtaaaggggctaaaaaccccggaagtcttcctgatccatggacccgatgaacatagacacaggtttgtttatttcagaagttctggtactaacatttggcagagcaaggtacggtgattggatcgaatctgtc

SEQ ID NO: 39 - DNA, 311 bp, Homo sapiens
tctcttgtaggccttgtggcatatttgaaataaaatagcttctgttattcagctctggctttcaggctagaggaagagcagaagatgaggccagaaattgctgggttaaaaccagcaaatccacccgtcttaccagctcctcagaaggcggacaccgaccctgagaacttacctgagatttcgtcacaggtactttgagcaaatggtgtggggttataactggggttcaatccagaggcatttcaaaccactgaagattgagaagaattattttcctttaccatttattaccacgtgtttacctctctctc

SEQ ID NO: 40 - DNA, 219 bp, unknown
  Gene sequence of the triple knocked-out cell strain MAN1A1/A2&B1 TKO2
ttctcttgtagggcttgtggcatatttgaaataaaatagcttctgttattcagctctggctttcaggctagaggaagagcagaagatgaggccagaaattgctgggttaaaaccagcaaacttacctgagatttcgtcacaggtactttgagcaaatggtgtggggttataactggggttcaatccagaggcatttcaaaccactgaagatgagaagaa

SEQ ID NO: 41 - DNA, 1131 bp, unknown
  DNA sequence for expressing alpha-lysosomal lipase to be inserted in the expression vector
gggaaactgacagctttggatcctgaaacaaacatgaatgtgagtgaaattatctcttactggggattccctagtgaggaatacctagttgagacagaagatggatatattctgtgccttaaccgaattcctcatgggaggaagaaccattctgacaaaggtcccaaaccagttgtcttcctgcaacatggcttgctggcagattctagtaactgggtcacaaaccttgccaacagcagcctgggcttcattcttgctgatgctggttttgacgtgtggatgggcaacagcagaggaaatacctggtctcggaaacataagacactctcagtttctcaggatgaattctgggctttcagttatgatgagatggcaaaatatgacctaccagcttccattaacttcattctgaataaaactggccaagaacaagtgtattatgtgggtcattctcaaggcaccactataggttttatagcattttcacagatccctgagctggctaaaaggattaaaatgttttttgccctgggtcctgtggcttccgtcgccttctgtactagccctatggccaaattaggacgattaccagatcatctcattaaggacttatttggagacaaagaatttcttccccagagtgcgtttttgaagtggctgggtacccacgtttgcactcatgtcatactgaaggagctctgtggaaatctctgttttcttctgtgtggatttaatgagagaaatttaaatatgtctagagtggatgtatatacaacacattctcctgctggaacttctgtgcaaaacatgttacactggagccaggctgttaaattccaaaagtttcaagcctttgactggggaagcagtgccaagaattattttcattacaaccagagttatcctcccacatacaatgtgaaggacatgcttgtgccgactgcagtctggagcgggggtcacgactggcttgcagatgtctacgacgtcaatatcttactgactcagatcaccaacttggtgttccatgagagcattccggaatgggagcatcttgacttcatttggggcctggatgccccttggaggctttataataaaattattaatctaatgaggaaatatcagtaa

SEQ ID NO: 42 - DNA, 1197 bp, unknown
  DNA sequence for expressing alpha-lysosomal galactosidase to be inserted in the expression vector
ctggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 43 - DNA, 2100 bp, Homo sapiens
atggctgcctgcgagggcaggagaagcggagctctcggttcctctcagtcggacttcctgacgccgccagtgggcggggccccttgggccgtcgccaccactgtagtcatgtacccaccgccgccgccgccgcctcatcgggacttcatctcggtgacgctgagctttggcgagaactatgacaacagcaagagttggcggcggcgctcgtgctggaggaaatggaagcaactgtcgagattgcagcggaatatgattctcttcctccttgcctttctgcttttctgtggactcctcttctacatcaacttggctgaccattggaaagctctggctttcaggctagaggaagagcagaagatgaggccagaaattgctgggttaaaaccagcaaatccacccgtcttaccagctcctcagaaggcggacaccgaccctgagaacttacctgagatttcgtcacagaagacacaaagacacatccagcggggaccacctcacctgcagattagacccccaagccaagacctgaaggatgggacccaggaggaggccacaaaaaggcaagaagcccctgtggatccccgcccggaaggagatccgcagaggacagtcatcagctggaggggagcggtgatcgagcctgagcagggcaccgagctcccttcaagaagagcagaagtgcccaccaagcctcccctgccaccggccaggacacagggcacaccagtgcatctgaactatcgccagaagggcgtgattgacgtcttcctgcatgcatggaaaggataccgcaagtttgcatggggccatgacgagctgaagcctgtgtccaggtccttcagtgagtggtttggcctcggtctcacactgatcgacgcgctggacaccatgtggatcttgggtctgaggaaagaatttgaggaagccaggaagtgggtgtcgaagaagttacactttgaaaaggacgtggacgtcaacctgtttgagagcacgatccgcatcctgggggggctcctgagtgcctaccacctgtctggggacagcctcttcctgaggaaagctgaggattttggaaatcggctaatgcctgccttcagaacaccatccaagattccttactcggatgtgaacatcggtactggagttgcccacccgccacggtggacctccgacagcactgtggccgaggtgaccagcattcagctggagttccgggagctctcccgtctcacaggggataagaagtttcaggaggcagtggagaaggtgacacagcacatccacggcctgtctgggaagaaggatgggctggtgcccatgttcatcaatacccacagtggcctcttcacccacctgggcgtattcacgctgggcgccagggccgacagctactatgagtacctgctgaagcagtggatccagggcgggaagcaggagacacagctgctggaagactacgtggaagccatcgagggtgtcagaacgcacctgctgcggcactccgagcccagtaagctcacctttgtgggggagcttgcccacggccgcttcagtgccaagatggaccacctggtgtgcttcctgccagggacgctggctctgggcgtctaccacggcctgcccgccagccacatggagctggcccaggagctcatggagacttgttaccagatgaaccggcagatggagacggggctgagtcccgagatcgtgcacttcaacctttacccccagccgggccgtcgggacgtggaggtcaagccagcagacaggcacaacctgctgcggccagagaccgtggagagcctgttctacctgtaccgcgtcacaggggaccgcaaataccaggactggggctgggagattctgcagagcttcagccgattcacacgggtcccctcgggtggctattcttccatcaacaatgtccaggatcctcagaagcccgagcctagggacaagatggagagcttcttcctgggggagacgctcaagtatctgttcttgctcttctccgatgacccaaacctgctcagcctggatgcctacgtgttcaacaccgaagcccaccctctgcctatctggacccctgcctag

SEQ ID NO: 44 - DNA, 1962 bp, Homo sapiens
atgcccgtggggggcctgttgccgctcttcagcagccccgcgggcggcgtcctgggcggggggctcggcggcggcggtggcaggaaggggtcgggccccgccgccctccgcctgacggagaagttcgtgctgctgctggtattcagcgccttcatcacgctctgcttcggggcgatcttcttcctgccagactcctccaagctgctcagcggggtcctgttccactccagccccgccttgcagccggccgccgaccacaagcccgggcccggggcgcgcgccgaggacgcggccgaggggcgagcccggcgccgcgaggagggggcacccggggacccggaggccgccctggaggacaacttggccaggatccgcgaaaaccacgagcgggctctcagggaagccaaggagaccctgcagaagctgcccgaggagatccaaagagacatcctactggagaagaagaaggtggcccaggaccagctgcgtgacaaggcgccgttcagaggcctgcccccggtggacttcgtgcccccaatcggggtggagagccgggagcccgccgacgccgccatccgcgagaaaagggcaaagatcaaagagatgatgaaacatgcttggaataattataaaggttatgcctggggattaaatgaactcaaacctatatcaaaaggaggccattcaagcagtttgtttggtaacatcaaaggagcaactatagtagatgccctggatacactttttattatggaaatgaaacatgaatttgaagaagcaaaatcatgggttgaagaaaatttagattttaatgtgaatgctgaaatttctgtctttgaagtaaatatacgctttgttggtggactactctcagcctactatctgtctggagaagagatttttcgaaagaaagcagtggaacttggggtaaaattgctacctgcatttcatactccctctggaataccttgggcattgctgaatatgaaaagtggtattggaaggaactggccctgggcctctggaggcagcagtattctggcagaatttggaaccctgcatttggagtttatgcacttgagccacttatcaggaaaccccatctttgctgaaaaggtaatgaatattcgaacagtactgaacaaactggaaaaaccacaaggcctttatcctaactatctgaatcccagtagtggacagtggggtcaacatcatgtatcagttggaggacttggagacagcttctatgagtatttgctgaaggcctggttaatgtctgacaagacagatctggaagctaagaagatgtattttgatgctgttcaggctatcgagactcatttgatccgcaagtctagcagcggactaacttatatcgcagagtggaaagggggcctcctggagcacaagatgggccacctgacctgcttcgcggggggcatgttcgcactcggggctgatgcagctcccgaaggcatggcccaacactaccttgaactcggggctgaaattgcccgtacttgtcatgaatcatataatcgaacatttatgaaactgggaccagaagctttcagatttgatggtggtgttgaagccatcgctacaagacaaaatgaaaaatactacatcttacggccagaagttatggagacttacatgtatatgtggagactgactcatgatccaaagtacaggaaatgggcctgggaagccgtagaggccttggaaaaccattgcagagtgaatggaggctattcaggcctaagggatgtttaccttcttcatgagagttatgatgatgtgcagcagagtttcttcctggcagagacattgaaatatttgtacctaatattttctgacgacgatcttcttccactggagcattggatcttcaatagcgaggcacatcttctccctatcctccctaaagataaaaaggaagttgaaatcagagaggaataa

SEQ ID NO: 45 - DNA, 1926 bp, Homo sapiens
atgactaccccagccctgctgcccctctctggacgtaggataccacctctgaacctggggccgccttccttcccacatcacagggctaccttgagactttctgagaagtttattcttctccttattcttagtgccttcatcactctgtgttttggggcattctttttccttccagactcttcaaaacacaaacgctttgatttgggtttagaagatgtgttaattccacatgtagatgccggtaaaggggctaaaaaccccggagtcttcctgatccatggacccgatgaacatagacacagggaagaggaagaacgtctgagaaataaaattcgagctgatcatgagaaggccttggaagaagcaaaagaaaaattaagaaagtcaagagaggaaattcgagcagaaattcagacagagaaaaataaggtagtccaagaaatgaagataaaagagaacaagccactgccaccagtccctattcccaaccttgtaggaatacgtggtggagacccagaagataatgacataagagagaaaagggaaaaaattaaagagatgatgaaacatgcttgggataactataggacatatgggtggggacataatgaactcagacctattgcaaggaaaggacactcccctaacatatttggaagttcacaaatgggtgctaccatagtagatgctttggataccctttatatcatgggacttcatgatgaattcctagatgggcaaagatggattgaagacaaccttgatttcagtgtgaattcagaggtgtctgtgtttgaagtcaacattcgatttattggaggcctacttgcagcatattacctatcaggagaggagatattcaagattaaagcagtgcaattggctgagaaactccttcctgcctttaacacacctactgggattccttgggcaatggtgaatttgaaaagtggagtagggcgaaactggggctgggcatctgcaggtagcagcattctggctgaatttggtacactacatatggagttcatccacctcagctacttgacaggggacctgacttactacaaaaaggttatgcacattcggaaactacttcagaaaatggatcgtccaaatggtctttatccaaattatttgaaccccagaacagggcgctggggtcagtatcatacatctgtcggtggcctgggagacagtttttatgaatacttactgaaagcatggttgatgtcagataaaacagaccatgaggcaagaaagatgtatgatgatgctattgaggctatagaaaaacatcttattaagaagtctcgtggaggtcttacctttattggagaatggaagaatgggcacttggaaaaaaagatggggcatttggcctgctttgctgggggaatgtttgcactaggagcagatggttccagagcagataaagctggtcattatttagagctaggggcagaaattgcacgtacttgtcatgagtcatatgacagaactgcattaaagctaggtcctgaatcattcaagtttgatggtgcagtggaggctgtggctgtccggcaggctgaaaagtattatatcctccgtccagaagtaattgaaacctattggtacctatggcgattcactcacgatccaagatacaggcagtggggctgggaagcagcactggccattgaaaagtattgccgagttaatggtgggttttctggagtcaaagatgtatattcctctactcctacacatgatgatgtacagcagagcttttttcttgctgaaacattaaaatatttgtatctgctgttctccggtgatgaccttttacctttagaccactgggtgtttaatacagaggctcaccctctgcctgtgttacatttagccaacaccacactttcaggtaatcctgctgttcgatga

SEQ ID NO: 46 - DNA, 1893 bp, Homo sapiens
atgctcatgaggaaagtgcccggcttcgtcccggcctccccgtgggggctgcggctgccgcagaagttcctcttcctcctcttcctctcgggcctggtcaccctgtgcttcggggccctcttcctgctgccccactcctctcgcctcaagcgcctcttcctggccccccggacccagcagcctggtctggaagtggtggctgaaatcgccggccatgccccggcccgcgagcaggagccgcctcccaacccggcccccgccgcgccggccccgggcgaggatgaccccagcagctgggccagtccccgccgcaggaaaggggggctgcggcgcacccgccccactggaccccgcgaggaggccacggcggcccggggcaatagcatcccggcctccaggcccggggacgagggcgtccctttccgctttgacttcaacgcattccggagccgtctccgccacccggtcctgggaacgagggccgatgagagtcaggagccccagagccaagtgcgagcccagcgggagaaaatcaaggagatgatgcagtttgcttggcagagctataagcgttatgcaatggggaaaaacgaactccgtccactaacaaaagatggctacgagggtaacatgttcggaggcctcagcggggcaacagtcattgactccctcgataccctctacctcatggagctgaaggaggagttccaggaggccaaggcctgggtgggagagagcttccacctgaacgtgagcggagaagcatccttgtttgaggtgaacatccgctacatcgggggactcctctcagccttctacctgacaggagaagaggtgttccgaataaaggccatcaggctgggagagaagctcctgccggcgttcaacacccccacgggaatcccaaagggcgtggtgagcttcaaaagtgggaactggggctgggccacagccggcagcagcagcatcttggcggagtttggatccctgcacttggaattcttacacctcactgaactctctggcaaccaggtcttcgctgaaaaggtcaggaacatccgcaaggtcctcaggaagatcgaaaagccctttggcctctaccccaacttcctcagcccagtgagtgggaactgggtgcaacaccatgtctcagttggaggactcggggacagtttttatgaatatttgatcaaatcctggttgatgtcgggcaagacagatatggaggctaaaaatatgtactacgaagccttggaggcgatagagacctacttgctgaatgtctctcccggggggctgacctacattgccgagtggcgaggggggattctggaccacaagatggggcacctggcctgtttctccgggggcatgatcgcccttggcgccgaggatgccaaggaagaaaagagggcccactaccgagagctcgcagcccagatcaccaagacgtgtcacgagtcatacgcccgctcagacaccaaacttgggcctgaggccttctggtttaactccggcagagaggccgtggccacccagctgagcgagagctactacatcctccggccagaggtggtggagagctacatgtacctgtggcgacagacccacaaccccatctacagggagtggggctgggaggtggtgctggccttggagaaatactgtcggacagaagccggtttctctgggatccaagacgtgtacagtagcacccccaaccacgacaacaagcagcagagcttctttctagcggagacactaaagtatctctatcttctgttctctgaagatgacttgctctccctggaagactgggtgttcaacaccgaggcccacccactcccggtgaaccactcagacagctccggcagagcctggggcagacactga